(19) United States
(12) Patent Application Publication
(10) Pub. No.: US 2004/0043030 A1
(43) Pub. Date: Mar. 4, 2004

(54) Polymeric delivery systems

(75) Inventors: Gary L. Griffiths, Morristown, NJ (US); David M. Goldenberg, Medham, NJ (US); Hans J. Hansen, Picayune, MS (US)

Correspondence Address: Stephen B. Maebius, Foley & Lardner, Washington Harbour, 3000 K Street, N.W., Suite 500, Washington, DC, 20007-5143, US

(73) Assignee: Immunomedics, Inc.

(21) Appl. No.: 10/456,580

(22) Filed: Jun. 9, 2003

Related U.S. Application Data
(63) Continuation-in-part of application No. 10/209,592, filed on Jul. 31, 2002.
(60) Provisional application No. 60/308,605, filed on Jul. 31, 2001.

(51) Int. Cl. G01N 33/574; A61K 39/395

(52) U.S. Cl. 424/178.1; 424/155.1; 435/7.23

(57) ABSTRACT

The present invention relates to a method of targeting an agent towards a targeting site in a tissue comprising administering a multi-specific antibody or antibody fragment comprising a targeting arm and a capture arm that binds to a polymer conjugate, and administering a polymer conjugate to the tissue. The present invention also relates to a kit for targeting a target site within a comprising a multi-specific antibody or antibody fragment comprising a targeting arm and a capture arm that binds to a polymer conjugate, and a polymer conjugate.

### FIELD OF THE INVENTION

The present invention relates to a method of targeting an agent towards a targeting site in a tissue comprising administering a multi-specific antibody or antibody fragment comprising a targeting arm and a capture arm that binds to a polymer conjugate, and administering a polymer conjugate to the tissue. The present invention also relates to a kit for targeting a target site within a tissue comprising a multi-specific antibody or antibody fragment comprising a targeting arm and a capture arm that binds to a polymer conjugate, and a polymer conjugate.

### BACKGROUND OF THE INVENTION

Among the current approaches in anti-cancer therapy that seek to improve therapeutic outcomes is the attachment of drugs to long-circulating polymers. Long-circulating polymers extend the half-life of the drug, prodrug or therapeutic agent in the blood, and generally allow for a greater proportion of the agent to reach the tumor site. Additionally, the tumor microenvironment enables macromolecules, including polymers, to accrete preferentially, allowing more of the therapeutic agent to reach the targeted site.

However, with increased half-life may come increased toxicity from the drug that is appended to the polymer, resulting is increased side effects that may negatively impact chemotherapy. Additionally, the longer-circulating polymer may itself elicit an immune response from the patient such that the polymer and the drug to which it is attached are bound by naturally occurring antibodies, and become ineffective.

To overcome these and other problems associated with polymer-drug conjugate therapy, the current invention relates to a method of further increasing the amount of drug-polymer conjugate that can be localized and retained at a tumor site. The method depends on the pre-injection of a multi-specific targeting agent, such as a multi-specific antibody, that has one arm directed against the cancer, and one arm directed against a hapten. Typically, agents useful in the current invention have a general formula comprising a (recognition hapten)ₙ-(polymer backbone)-(drug or prodrug therapy moiety)ₘ, or a polymer backbone-(drug or prodrug therapy moiety)ₘ wherein n and m are integers reflecting different substitution levels on the polymer backbone for the respective species. The polymer-drug conjugate is used after the cancer has been pre-targeted with a multi-specific antibody. In the former case, one arm recognizes the hapten. In the latter case, one arm of the bispecific is directed against some or all of the polymer backbone, or some or all of the appended drug.

The methods disclosed herein may be used for therapeutic or diagnostic purposes. Additionally, a system where the multi-specific antibody recognizes a polymer conjugate will be extremely versatile in a wide array of applications as will be apparent from the description that follows.

### SUMMARY OF THE INVENTION

The present invention relates to a method for targeting an agent towards a target site in a tissue, comprising (a) administering to a tissue a multi-specific antibody (msAb) or multi-specific antibody fragment, comprising a targeting arm that binds to an antigen on the target site, and a capture arm that binds to a polymer conjugate; and (b) administering to the tissue a polymer conjugate that binds to the capture arm, with the polymer conjugate comprising a polymer conjugated to an agent selected from the group consisting of a therapeutic agent, a peptide, an enzyme and a labeled ligand.

The present invention also relates to a kit, useful for targeting a target site in a tissue or tissue sample, comprising, (a) a multi-specific antibody or antibody fragment comprising a targeting arm that binds to an antigen on said target site, and a capture arm that binds to a polymer conjugate or hapten-polymer conjugate; and (b) a polymer conjugate that binds to the capture arm, with the polymer conjugate comprising a polymer conjugated to an agent selected from the group consisting of a therapeutic agent, a peptide, an enzyme and a labeled ligand.

Also contemplated in the present invention is a method for diagnosing or treating a disease or disorder comprising: (a) administering to a tissue a multi-specific antibody or antibody fragment, comprising a targeting arm that binds to an antigen on said target site, and a capture arm that binds to a polymer conjugate; and (b) administering to said tissue a polymer conjugate that binds to said capture arm, said polymer conjugate comprising a polymer conjugated to said agent selected from the group consisting of a therapeutic agent, a diagnostic agent, a peptide, an enzyme and a labeled ligand. Preferably, the disease or disorder is selected from the group consisting of a cancer, cardiovascular lesion, an inflammatory disease and an infectious disease. More preferably, the disease or disorder is an autoimmune disease. Still preferred, the disease or disorder is a CEA-expressing tumor or a CD20-expressing malignancy, such as a B-cell lymphoma or leukemia. Also preferred, the compositions and methods of the present invention can be used for therapy and/or diagnosis or imaging for cardiovascular lesions (infarcts, clots, emboli, atherosclerotic plaques, ischemia), other pathological lesions (e.g., amyloid in amyloidosis and in Alzheimer's disease), cancers (e.g., leukemias, lymphomas, sarcomas, melanomas, carcinomas, gliomas, skin cancers), infectious diseases (e.g., bacterial, rickettsial, fungal, parasitical, and viral pathogens), inflammation (e.g., autioimmune diseases, such as rheumatoid arthritis, systemic erythematosis, multiple sclerosis), displaced or ectopic normal tissues and cells (e.g., endometrium, thymus, spleen, parathyroid), normal tissue ablation (e.g., bone marrow, spleen).

Still preferred, the antigen is selected from the group consisting of carcinoembryonic antigen (CEA), colon-specific antigen-p, HER-2/neu, epidermal growth factor receptor (EGFR), VEGF, placental growth factor (PLGF), tenascin, EGP-1, EGP-2, CD19, CD20, CD22, CD21, CD23, CD30, CD33, CD45, CD80, and CD74. α-fetoprotein, A3, A33, CA-125, colon-specific antigen-p (CSAp), folate receptor, HLA-DR, human chorionic gonadrotropin, Ia, IL-2, insulin-like growth factor, KS-1, Le(y), MAGE, MUC1, MUC2, MUC3, MUC4, NCA66, necrosis antigens, PAM-4, prostatic acid phosphatase (PAP), prostate specific antigen (PSA), PSMA, S100, T101, TAC, IL-6 and TAG-72.

Also contemplated in the present invention is a method for photodynamic diagnosis or treatment of a disease or disorder comprising: (a) administering to a tissue a multi-specific antibody or antibody fragment, comprising a targeting arm that binds to an antigen on said target site, and a capture arm that binds to a polymer conjugate; and (b) administering to said tissue a polymer conjugate that binds to said capture arm, said polymer conjugate comprising a polymer conjugated to a diagnostic or therapeutic agent. Preferably, the therapeutic agent is a photosensitizer, such as a dihematoporphyrin, benzoporphyrin monoacid ring A, tin etiopurpurin, sulfonated aluminum phthalocyanine, and lutetium texaphyrin.

### BRIEF DESCRIPTION OF THE DRAWINGS

N/A

### DETAILED DESCRIPTION OF PREFERRED EMBODIMENTS

The current invention relates to a method for targeting an agent towards a target site in a tissue, comprising (a) administering to the tissue a multi-specific antibody (msAb) or multi-specific antibody fragment, comprising a targeting arm that binds to an antigen on said target site, and a capture arm that binds to a polymer conjugate; and (b) administering to the tissue a polymer conjugate that binds to the capture arm, the polymer conjugate comprising a polymer conjugated to the agent selected from the group consisting of a therapeutic agent, a peptide, an enzyme and a labeled ligand. Preferably, the polymer conjugate of the current invention has the general formula (polymer-backbone)-(agent)ₘ, where m is an integer, including 0.

As used herein, the term tissue is used to mean a tissue as one of ordinary skill in the art would understand it to mean. As envisioned in the current application, tissue is also used to mean individual or groups of cells, or cell cultures, of a bodily tissue or fluid (e.g. blood cells). Furthermore, the tissue may be within a subject, or biopsied or removed from a subject. The tissue may also be a whole or any portion of a bodily organ. Additionally, the tissue may be “fresh” in that the tissue would be recently removed from a subject without any preservation steps between the excision and the methods of the current invention. The tissue may also have been preserved by such standard tissue preparation techniques including, but not limited to, freezing, quick freezing, paraffin embedding and tissue fixation, prior to application of the methods of the current invention.

As used herein, the terms patient or subject are used interchangeably and are used to mean any animal, preferably a mammal, including humans and non-human primates.

As used herein, the term target site is used to mean a site at the locus of the tissue towards which any type of agent or compound may be directed. Locus can be used to mean any portion of the tissue or cells itself, including normal and/or pathogenic portions. Locus can also mean the area that surrounds the tissue that may contain a causal or symptomatic agent of the diseased tissue. As such, the methods of the present invention can be used to target an agent toward a target site in a tissue, including a tissue afflicted with a particular disease or disorder.

The target site may be the entire tissue, or may be a portion of the tissue, such as blood vessels within a tumor, or may be individual or groups of cells, in vivo or in vitro, or in situ, that make up the tissue. For example, the antigen on the target site may be associated with the vascular endothelium of tumors, including tenascin, vascular endothelium growth factor (VEGF), and placental growth factor (PLGF). It also can be a molecule or molecular subunit that accretes at the locus of the target tissue. Additionally, the target site can be associated with a pathogen in proximity to a diseased tissue, thus the target site does not necessarily have to be directly contacting or integrated with the cell or tissue. The phrases targeted site and targeted tissue are used interchangeably herein.

The current invention utilizes multi-specific antibodies (msAbs) to direct the polymer conjugate to a target site within a tissue. As used herein, multi-specific antibodies have more than one specificity, or more than one valency, such that the msAbs of the invention bind to or recognize more than one antigen or epitope. For example, a bi-specific antibody of the current invention would include an antibody where each arm of the immunoglobulin recognizes or binds to a separate epitope or hapten. Multi-specific antibodies of the current invention also encompass specificities higher than bi-specific, such as, but not limited to, tri-specific or tetra-specific antibodies. For example, tri-specific antibodies may comprise an antibody with two arms directed towards two different cellular antigens, and a third arm directed towards a hapten or drug. As used herein, multi-specific antibodies also include antibodies with more than one valency. For example, an antibody encompassed in the current invention may be comprised of two arms directed towards a single cellular epitope, and a third arm directed towards a hapten or drug, such that the antibody is bi-specific, yet tri-valent. Furthermore, the target arm or arms of the msAb may be directed to two or more distinct epitopes on the target tissue, and the capture arm or arms may be directed to two or more distinct haptens on the polymer conjugate.

As the current invention contemplates, msAbs encompass antibodies multi-specific antibody fragments. The antibody fragments are antigen binding portions of an antibody, such as F(ab′)₂, F(ab)₂, Fab′, Fab, and the like. The antibody fragments bind to the same antigen that is recognized by the intact antibody. For example, an anti-CD22 monoclonal antibody fragment binds to an epitope of CD22. The msAbs of the present invention include, but are not limited to, IgG×IgG, IgG×F(ab′)₂, IgG×Fab′, IgG×scFv, F(ab′)₂×F(ab′)₂, Fab′×F(ab′)₂, Fab′×Fab′, Fab′×scFv and scFv×scFv bi-specific monoclonal antibodies (bismAbs). Also, species such as scFv×IgG×scFv and Fab′×IgG×Fab′, scFv×F(ab′)₂×scFv and Fab′×F(ab′)₂×Fab′are included. Most preferably, site-specific attachment sites on the IgG or F(ab′)₂ of one or both monoclonal antibodies (mAbs) can be utilized, such as an engineered carbohydrate or an engineered or liberated free thiol group. Since these mAbs are dimeric they can be coupled with two moles of the second mAb. For instance, a mAb directed towards carcinoembryonic antigen (CEA), anti-CEA F(ab′)₂, having an engineered light-chain carbohydrate can be oxidized and converted using a hydrazide-maleimide cross-linker to a derivatized anti-CEA F(ab′)₂ having at least one pendant maleimide group per each light chain. This species is coupled to an anti-chelate Fab′-SH at a 1:2 molar ratio, at least, such that an anti-chelate-Fab′×anti-CEA-F(ab′)₂-anti-chelate Fab′ conjugate is produced. The resultant msAb is bivalent with respect to the target tissue and the polymer conjugate. It is further understood that the use of the term “msAb” in the present disclosure encompasses multi-specific antibodies and multi-specific antibody fragments.

The term “antibody fragment” also includes any synthetic or genetically engineered protein that acts like an antibody by binding to a specific antigen to form a complex. For example, antibody fragments include isolated fragments, “Fv” fragments, consisting of the variable regions of the heavy and light chains, recombinant single chain polypeptide molecules in which light and heavy chain variable regions are connected by a peptide linker (“sFv proteins”), and minimal recognition units consisting of the amino acid residues or related peptides that mimic the hypervariable region.

The msAbs of the current invention may be monoclonal or polyclonal in nature, but preferably monoclonal. Furthermore, the targeting arm and the capture arm of the msAb may be monoclonal or polyclonal in nature. Preferably, either the target arm or the capture arm is monoclonal. Most preferably, the target arm and the capture arm are both monoclonal.

The msAb of the current invention may be engineered to possess a label. Examples of labels that the msAb may possess include, but are not limited to, a labeling ligand such as the biotin-streptavidin complex and radioisotopes. Advantageously, the msAb of the current invention is radiolabeled to facilitate tracking of localization and clearance.

One or both of the targeting arm and the capture arm of the msAb may be chimeric, human or humanized.

As used herein, “targeting arm” is used to mean the portion of the msAb that recognizes and/or binds to an antigen present at the locus of the targeted tissue. The antigen may be attached externally to a cell or tissue, or part of the cell-surface membrane, or may be a glycosyl-phosphatidylinositol (GPI)-anchored protein or may be internal to a cell. Additionally, the antigen may be associated with fluids including, but not limited to, any part of whole blood, lymphatic fluid or cerebrospinal fluid. Furthermore, the antigen may be present in, accreted by or secreted or released by normal, abnormal, diseased or necrotic cells or tissue. Therefore, the antigen may be present in a tissue afflicted by a human disorder such as cancer, infectious disease or inflammatory disease, including an autoimmune disease. Exemplary autoimmune diseases include myasthenia gravis, lupus nephritis, lupus erythematosus, and rheumatoid arthritis, Class II and Class III autoimmune diseases, such as celiac disease, type I diabetes, immune-mediated thrombocytopenias, such as acute idiopathic thrombocytopenic purpura and chronic idiopathic thrombocytopenic purpura, dermatomyositis, Sjögren's syndrome, multiple sclerosis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, sarcoidosis, ulcerative colitis, erythema multiforme, IgA nephropathy, polyarteritis nodosa, ankylosing spondylitis, Goodpasture's syndrome, thromboangitis ubiterans, Sjogren's syndrome, primary biliary cirrhosis, Hashimoto's thyroiditis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis and fibrosing alveolitis.

Additionally, the antigen may be present on pathogens, including, but not limited to viruses, bacteria and/or prions that are located at the locus of the targeted tissue. Thus the antigen does not necessarily have to be directly contacting or integrated with the cell. The antigen may have specific characteristics, such as a distinct cell-surface-associated antigen, or it may have general characteristics that are shared by more than one tissue or cell type. For example, β1-integrin is an extracellular cell adhesion molecule shared by a variety of normal or diseased tissue that is antigenic and would be considered an antigen at the locus of a target site within the context of the current invention. Examples of antigens include, but are not limited to, MHC complex components, receptors and tumor/carcinoma-associated antigens. Specifically, such antigens include carcinoembryonic antigen (CEA), HER-2/neu, epidermal growth factor receptor (EGFR), VEGF, placental growth factor (PLGF), tenascin, EGP-1, EGP-2, CD19, CD20, CD22, CD21, CD23, CD30, CD33, CD45, CD80, and CD74. α-fetoprotein, A3, A33, CA-125, colon-specific antigen-p (CSAp), folate receptor, HLA-DR, human chorionic gonadrotropin, Ia, IL-2, insulin-like growth factor, KS-1, Le(y), MAGE, MUC1, MUC2, MUC3, MUC4, NCA66, necrosis antigens, PAM-4, prostatic acid phosphatase (PAP), prostate specific antigen (PSA), Pr1, PSMA, S100, T101, TAC, IL-6 and TAG-72. As such, the methods of the present invention can be used to target an agent toward a target site in a malignant tissue. For example, the agent can be targeted to CEA-expressing tumors or CD20-expressing malignancies such as B-cell lymphomas and leukemias.

As used herein, the “capture arm” is used to mean the portion of the msAb that recognizes and binds to the polymer conjugate. The capture arm may recognize the polymeric backbone of the polymer conjugate directly, or the agent conjugated to the polymer backbone, or a hapten bound to the polymer-drug conjugate.

Antibodies to polymer backbones comprised of, for example, peptides, are generated by well-known methods for Ab production. For example, injection of an immunogen, such as (peptide)ₙ-KLH (n=1-30) in complete Freund's adjuvant, followed by two subsequent injections of the same immunogen suspended in incomplete Freund's adjuvant into immunocompetent animals, is followed three days after an i.v. boost of antigen, by spleen cell harvesting. Harvested spleen cells are then fused with Sp2/0-Ag14 myeloma cells and culture supernatants of the resulting clones analyzed for anti-peptide reactivity using a direct-binding ELISA. Fine specificity of generated Abs can be analyzed for by using peptide fragments of the original immunogen. These fragments can be prepared readily using an automated peptide synthesizer. For Ab production, enzyme-deficient hybridomas are isolated to enable selection of fused cell lines. This technique also can be used to raise antibodies to one or more of the chelates comprising the polymer conjugate, e.g., In(III)-DTPA chelates. Monoclonal mouse antibodies to an In(III)-di-DTPA are known in the art, for example U.S. Pat. No. 5,256,395.

After the initial raising of antibodies to the immunogen, the antibodies can be sequenced and subsequently prepared by recombinant techniques. Humanization and chimerization of murine antibodies and antibody fragments are well known to those skilled in the art. For example, humanized monoclonal antibodies are produced by transferring mouse complementary determining regions from heavy and light variable chains of the mouse immunoglobulin into a human variable domain, and then, substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by the publication of Orlandi et al., Proc. Nat'l Acad. Sci. USA 86: 3833 (1989), which is incorporated by reference in its entirety. Techniques for producing humanized mAbs are described, for example, by Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun. 150: 2844 (1993), each of which is hereby incorporated by reference.

Alternatively, fully human antibodies can be obtained from transgenic non-human animals. See, e.g., Mendez et al., Nature Genetics, 15: 146-156 (1997); U.S. Pat. No. 5,633,425. For example, human antibodies can be recovered from transgenic mice possessing human immunoglobulin loci. The mouse humoral immune system is humanized by inactivating the endogenous immunoglobulin genes and introducing human immunoglobulin loci. The human immunoglobulin loci are exceedingly complex and comprise a large number of discrete segments which together occupy almost 0.2% of the human genome. To ensure that transgenic mice are capable of producing adequate repertoires of antibodies, large portions of human heavy- and light-chain loci must be introduced into the mouse genome. This is accomplished in a stepwise process beginning with the formation of yeast artificial chromosomes (YACs) containing either human heavy- or light-chain immunoglobulin loci in germline configuration. Since each insert is approximately 1 Mb in size, YAC construction requires homologous recombination of overlapping fragments of the immunoglobulin loci. The two YACs, one containing the heavy-chain loci and one containing the light-chain loci, are introduced separately into mice via fusion of YAC-containing yeast spheroblasts with mouse embryonic stem cells. Embryonic stem cell clones are then microinjected into mouse blastocysts. Resulting chimeric males are screened for their ability to transmit the YAC through their germline and are bred with mice deficient in murine antibody production. Breeding the two transgenic strains, one containing the human heavy-chain loci and the other containing the human light-chain loci, creates progeny which produce human antibodies in response to immunization.

Unrearranged human immunoglobulin genes also can be introduced into mouse embryonic stem cells via microcell-mediated chromosome transfer (MMCT). See, e.g., Tomizuka et al., Nature Genetics, 16: 133 (1997). In this methodology microcells containing human chromosomes are fused with mouse embryonic stem cells. Transferred chromosomes are stably retained, and adult chimeras exhibit proper tissue-specific expression.

As an alternative, an antibody or antibody fragment of the present invention may be derived from human antibody fragments isolated from a combinatorial immunoglobulin library. See, e.g., Barbas et al., METHODS: A Companion to Methods in Enzymology 2: 119 (1991), and Winter et al., Ann. Rev. Immunol. 12: 433 (1994), which are incorporated by reference. Many of the difficulties associated with generating monoclonal antibodies by B-cell immortalization can be overcome by engineering and expressing antibody fragments in E. coli, using phage display. To ensure the recovery of high affinity, monoclonal antibodies a combinatorial immunoglobulin library must contain a large repertoire size. A typical strategy utilizes mRNA obtained from lymphocytes or spleen cells of immunized mice to synthesize cDNA using reverse transcriptase. The heavy- and light-chain genes are amplified separately by PCR and ligated into phage cloning vectors. Two different libraries are produced, one containing the heavy-chain genes and one containing the light-chain genes. Phage DNA is islolated from each library, and the heavy- and light-chain sequences are ligated together and packaged to form a combinatorial library. Each phage contains a random pair of heavy- and light-chain cDNAs and upon infection of E. coli directs the expression of the antibody chains in infected cells. To identify an antibody that recognizes the antigen of interest, the phage library is plated, and the antibody molecules present in the plaques are transferred to filters. The filters are incubated with radioactively labeled antigen and then washed to remove excess unbound ligand. A radioactive spot on the autoradiogram identifies a plaque that contains an antibody that binds the antigen. Cloning and expression vectors that are useful for producing a human immunoglobulin phage library can be obtained, for example, from STRATAGENE Cloning Systems (La Jolla, Calif.).

A similar strategy can be employed to obtain high-affinity scFv. See, e.g., Vaughn et al., Nat. Biotechnol., 14: 309-314 (1996). An scFv library with a large repertoire can be constructed by isolating V-genes from non-immunized human donors using PCR primers corresponding to all known V heavy-chain (V_{H}) and V light-chains (V_{κ} and V_{λ}) gene families. Following amplification, the V_{κ} and V_{λ} pools are combined to form one pool. These fragments are ligated into a phagemid vector. The scFv linker, (Gly₄-Ser₁)₃, is then ligated into the phagemid upstream of the V light-chain (V_{L}) fragment. The V_{H} and linker-V_{L} fragments are amplified and assembled on the J_{H} region. The resulting V_{H}-linker-V_{L} fragments are ligated into a phagemid vector. The phagemid library can be panned using filters, as described above, or using immunotubes (Nunc; Maxisorp). Similar results can be achieved by constructing a combinatorial immunoglobulin library from lymphocytes or spleen cells of immunized rabbits and by expressing the scFv constructs in P. pastoris. See, e.g., Ridder et al., Biotechnology, 13: 255-260 (1995). Additionally, following isolation of an appropriate scFv, antibody fragments with higher binding affinities and slower dissociation rates can be obtained through affinity maturation processes such as CDR3 mutagenesis and chain shuffling. See, e.g., Jackson et al., Br. J. Cancer, 78: 181-188 (1998); Osbourn et al., Immunotechnology, 2: 181-196 (1996).

The msAb can be prepared by techniques known in the art, for example, an anti-CEA tumor Ab and an anti-peptide Ab are both separately digested with pepsin to their respective F(ab′)_{2S}. The anti-CEA-Ab-F(ab′)₂ is reduced with cysteine to generate Fab′ monomeric units which are further reacted with a cross-linker such as bis(maleimido) hexane to produce Fab′-maleimide moieties. The anti-peptide Ab-F(ab′)₂ is reduced with cysteine and the purified, recovered anti-peptide Fab′-SH reacted with the anti-CEA-Fab′-maleimide to generate the Fab′×Fab′ bi-specific Ab. Alternatively, the anti-peptide Fab′-SH fragment may be coupled with the anti-CEA F(ab′)₂ to generate a F(ab′)₂×Fab′ construct, or with anti-CEA IgG to generate an IgG×Fab′ bi-specific construct. In one embodiment, the IgG×Fab′ construct can be prepared in a site-specific manner by attaching the anti-peptide Fab′ thiol group to anti-CEA IgG heavy-chain carbohydrate which has been periodate-oxidized, and subsequently activated by reaction with a commercially available hydrazide-maleimide cross-linker. The component Abs used can be chimerized or humanized by known techniques. A chimeric antibody is a recombinant protein that contains the variable domains and complementary determining regions derived from a rodent antibody, while the remainder of the antibody molecule is derived from a human antibody. Humanized antibodies are recombinant proteins in which murine complementarity determining regions of a monoclonal antibody have been transferred from heavy and light variable chains of the murine immunoglobulin into a human variable domain.

A variety of recombinant methods can be used to produce multi-specific antibodies and antibody fragments. For example, multi-specific antibodies and antibody fragments can be produced in the milk of transgenic livestock. See, e.g., Colman, A., Biochem. Soc. Symp., 63: 141-147, 1998; and U.S. Pat. No. 5,827,690. Two DNA constructs are prepared which contain, respectively, DNA segments encoding paired immunoglobulin heavy and light chains. The fragments are cloned into expression vectors which contain a promoter sequence that is preferentially expressed in mammary epithelial cells. Examples include, but are not limited to, promoters from rabbit, cow and sheep casein genes, the cow α-lactoglobulin gene, the sheep β-lactoglobulin gene and the mouse whey acid protein gene. Preferably, the inserted fragment is flanked on its 3′ side by cognate genomic sequences from a mammary-specific gene. This provides a polyadenylation site and transcript-stabilizing sequences. The expression cassettes are coinjected into the pronuclei of fertilized, mammalian eggs, which are then implanted into the uterus of a recipient female and allowed to gestate. After birth, the progeny are screened for the presence of both transgenes by Southern analysis. For the antibody to be present, both heavy and light chain genes must be expressed concurrently in the same cell. Milk from transgenic females is analyzed for the presence and functionality of the antibody or antibody fragment using standard immunological methods known in the art. The antibody can be purified from the milk using standard methods known in the art.

A chimeric Ab is constructed by ligating the cDNA fragment encoding the mouse light variable and heavy variable domains to fragment encoding the C domains from a human antibody. Because the C domains do not contribute to antigen binding, the chimeric antibody will retain the same antigen specificity as the original mouse Ab but will be closer to human antibodies in sequence. Chimeric Abs still contain some mouse sequences, however, and may still be immunogenic. A humanized Ab contains only those mouse amino acids necessary to recognize the antigen. This product is constructed by building into a human antibody framework the amino acids from mouse complementarity determining regions.

Other recent methods for producing msAbs include engineered recombinant Abs which have additional cysteine residues so that they crosslink more strongly than the more common immunoglobulin isotypes. See, e.g., FitzGerald et al., Protein Eng. 10(10): 1221-1225, 1997. Another approach is to engineer recombinant fusion proteins linking two or more different single-chain antibody or antibody fragment segments with the needed dual specificities. See, e.g., Coloma et al., Nature Biotech. 15:159-163, 1997. A variety of multi-specific fusion proteins can be produced using molecular engineering. In one form, the multi-specific fusion protein is monovalent, consisting of, for example, a scFv with a single binding site for one antigen and a Fab fragment with a single binding site for a second antigen. In another form, the multi-specific fusion protein is divalent, consisting of, for example, an IgG with two binding sites for one antigen and two scFv with two binding sites for a second antigen.

Functional multi-specific single-chain antibodies (mscAbs), also called diabodies, can be produced in mammalian cells using recombinant methods. See, e.g., Mack et al., Proc. Natl. Acad. Sci., 92: 7021-7025, 1995. For example, mscAbs are produced by joining two single-chain Fv fragments via a glycine-serine linker using recombinant methods. The V light-chain (V_{L}) and V heavy-chain (V_{H}) domains of two antibodies of interest are isolated using standard PCR methods. The V_{L} and V_{H} cDNA's obtained from each hybridoma are then joined to form a single-chain fragment in a two-step fusion PCR. The first PCR step introduces the (Gly₄-Ser₁)₃ linker, and the second step joins the V_{L} and V_{H} amplicons. Each single chain molecule is then cloned into a bacterial expression vector. Following amplification, one of the single-chain molecules is excised and sub-cloned into the other vector, containing the second single-chain molecule of interest. The resulting mscAb fragment is subcloned into an eukaryotic expression vector. Functional protein expression can be obtained by transfecting the vector into chinese hamster ovary cells. Multi-specific fusion proteins are prepared in a similar manner. Multi-specific single-chain antibodies and multi-specific fusion proteins are included within the scope of the present invention.

Multi-specific fusion proteins linking two or more different single-chain antibodies or antibody fragments are produced in similar manner as discussed above. Recombinant methods can be used to produce a variety of fusion proteins. For example a fusion protein comprising a Fab fragment derived from a humanized monoclonal anti-CEA antibody and a scFv derived from a murine anti-diDTPA can be produced. A flexible linker, such as (GGGS)₃, which is a trimer of glycyl-glycyl-glycyl-serine connects the scFv to the constant region of the heavy chain of the anti-CEA antibody. Alternatively, the scFv can be connected to the constant region of the light chain of hMN-14. Appropriate linker sequences necessary for the in-frame connection of the heavy chain Fd to the scFv are introduced into the V_{λ} and V_{κ} domains through PCR reactions. The DNA fragment encoding the scFv is then ligated into a staging vector containing a DNA sequence encoding the CH1 domain. The resulting scFv-CH1 construct is excised and ligated into a vector containing a DNA sequence encoding the VH region of an anti-CEA antibody. The resulting vector can be used to transfect mammalian cells for the expression of the multi-specific fusion protein.

Large quantities of bscAb and fusion proteins can be produced using Escherichia coli expression systems. See, e.g., Zhenping et al., Biotechnology, 14: 192-196, 1996. A functional bscAb can be produced by the coexpression in E. coli of two “cross-over” scFv fragments in which the V_{L} and V_{H} domains for the two fragments are present on different polypeptide chains. The V_{L} and V_{H} domains of two antibodies of interest are isolated using standard PCR methods. The cDNA's are then ligated into a bacterial expression vector such that C-terminus of the V_{L} domain of the first antibody of interest is ligated via a linker to the N-terminus of the V_{H} domain of the second antibody. Similarly, the C-terminus of the V_{L} domain of the second antibody of interest is ligated via a linker to the N-terminus of the V_{H} domain of the first antibody. The resulting dicistronic operon is placed under transcriptional control of a strong promoter, e.g., the E. coli alkaline phosphatase promoter which is inducible by phosphate starvation. Alternatively, single-chain fusion constructs have successfully been expressed in E. coli using the lac promoter and a medium consisting of 2% glycine and 1% Triton X-100. See, e.g., Yang et al., Appl. Environ. Microbiol., 64: 2869-2874, 1998. An E. coli, heat-stable, enterotoxin II signal sequence is used to direct the peptides to the periplasmic space. After secretion, the two peptide chains associate to form a non-covalent heterodimer which possesses both antigen binding specificities. The bscAb is purified using standard procedures known in the art, e.g., Staphylococcal protein A chromatography.

Functional bscAb and fusion proteins also can be produced in the milk of transgenic livestock. See, e.g., Colman, A., Biochem. Soc. Symp., 63: 141-147, 1998; U.S. Pat. No. 5,827,690. The bscAb fragment, obtained as described above, is cloned into an expression vector containing a promoter sequence that is preferentially expressed in mammary epithelial cells. Examples include, but are not limited to, promoters from rabbit, cow and sheep casein genes, the cow α-lactoglobulin gene, the sheep β-lactoglobulin gene and the mouse whey acid protein gene. Preferably, the inserted bscAb is flanked on its 3′ side by cognate genomic sequences from a mammary-specific gene. This provides a polyadenylation site and transcript-stabilizing sequences. The expression cassette is then injected into the pronuclei of fertilized, mammalian eggs, which are then implanted into the uterus of a recipient female and allowed to gestate. After birth, the progeny are screened for the presence of the introduced DNA by Southern analysis. Milk from transgenic females is analyzed for the presence and functionality of the bscAb using standard immunological methods known in the art. The bscAb can be purified from the milk using standard methods known in the art. Transgenic production of bscAb in milk provides an efficient method for obtaining large quantities of bscAb.

Functional bscAb and fusion proteins also can be produced in transgenic plants. See, e.g., Fiedler et al., Biotech., 13: 1090-1093, 1995; Fiedler et al., Immunotechnology, 3; 205-216, 1997. Such production offers several advantages including low cost, large scale output and stable, long term storage. The bscAb fragment, obtained as described above, is cloned into an expression vector containing a promoter sequence and encoding a signal peptide sequence, to direct the protein to the endoplasmic recticulum. A variety of promoters can be utilized, allowing the practitioner to direct the expression product to particular locations within the plant. For example, ubiquitous expression in tobacco plants can be achieved by using the strong cauliflower mosaic virus 35S promoter, while organ specific expression is achieved via the seed specific legumin B4 promoter. The expression cassette is transformed according to standard methods known in the art. Transformation is verified by Southern analysis. Transgenic plants are analyzed for the presence and functionality of the bscAb using standard immunological methods known in the art. The bscAb can be purified from the plant tissues using standard methods known in the art.

Additionally, transgenic plants facilitate long term storage of bscAb and fusion proteins. Functionally active scFv proteins have been extracted from tobacco leaves after a week of storage at room temperature. Similarly, transgenic tobacco seeds stored for 1 year at room temperature show no loss of scFv protein or its antigen binding activity.

Functional bscAb and fusion proteins also can be produced in insect cells. See, e.g., Mahiouz et al., J. Immunol. Methods, 212: 149-160 (1998). Insect-based expression systems provide a means of producing large quantities of homogenous and properly folded bscAb. The baculovirus is a widely used expression vector for insect cells and has been successfully applied to recombinant antibody molecules. See, e.g., Miller, L. K., Ann. Rev. Microbiol., 42: 177 (1988); Bei et al., J. Immunol. Methods, 186: 245 (1995). Alternatively, an inducible expression system can be utilized by generating a stable insect cell line containing the bscAb construct under the transcriptional control of an inducible promoter. See, e.g., Mahiouz et al., J. Immunol. Methods, 212: 149-160 (1998). The bscAb fragment, obtained as described above, is cloned into an expression vector containing the Drosphila metallothionein promoter and the human HLA-A2 leader sequence. The construct is then transfected into D. melanogaster SC-2 cells. Expression is induced by exposing the cells to elevated amounts of copper, zinc or cadmium. The presence and functionality of the bscAb is determined using standard immunological methods known in the art. Purified bscAb is obtained using standard methods known in the art.

The polymers used in the current invention are meant to provide a backbone upon which a single molecule or a plurality of molecules of an agent may be attached. A plurality of molecules, as used herein, means more than one molecule. Additionally, more than one kind of agent may be attached to the same backbone to allow delivery of multiple agents on a single polymer. Agents that may be attached to the same polymeric backbone may differ in properties including, but not limited to, stereochemistry, chemical formula, radioactive isotope number, atomic weight, half-life, activity, specificity, energy of activation, radioactivity and potency.

Exemplary polymers and polymer backbones of the invention are polymers of single amino acids such as polylysine, polyglutamic (E; single letter code) and aspartic acids (D), including D-amino acid analogs of the same. In one embodiment, unnatural amino acids, e.g., D-amino acids, are incorporated into the backbone structure to ensure that, when used with the final msAb/polymer conjugate system, the arm of the msAb which recognizes the polymer conjugate is completely specific. Co-polymers, as used herein, means polymers of two or more amino acids, including, but not limited to, polymers of three amino acids, polymers of four amino acids and polymers of five amino acids. Co-polymers such as poly(Lys-Glu) {poly[KE]} are especially useful, when such co-polymers are selected with the building blocks in desirable ratios to each other. These ratios may be advantageously from 1:10 to 10:1, in the case of poly[KE] or poly[KD]. More complex co-polymers based on amino acid building blocks such as poly(Lys-Ala-Glu-Tyr) (KAEY; 5:6:2:1) may also be employed. The molecular weight of the polymer used is generally within the range 1,000 to 100,000 Daltons. Amino acid building blocks are chosen not only for their ability to act as carriers for the recognition hapten and therapy agent, but also for the physical and biological properties that the individual building blocks can make to the overall polymer conjugates. For instance, a preferred polymer conjugate is one that retains adequate solubility even when multiply substituted with hydrophobic drug moieties. In the case of polypeptides this often means an abundance of charged residues being present. Another preferred property is engendered in a final polymer conjugate that retains a net negative charge at physiological pH, since agents with net positive charges can sometimes bind non-specifically to cells and tissues. In the case of polypeptides a preponderance of acidic residues such as aspartate and glutamate most readily satisfy this criteria. A third preferred property is that the polymer backbone is stable to serum enzymes such as esterases, and carboxy-and amino-peptidases. For this preference, polypeptides can incorporate D-amino acids, and will be acylated and amidated, at the N- and C-termini, respectively. In terms of preferred molecular weight ranges base polymer weights between 5,000 and 25,000 are especially preferred.

However, smaller polymer conjugates of completely defined molecular weight are also preferred within the scope of the invention. These may be produced as chemically defined entities by solid-phase peptide synthesis techniques, readily producing polypeptides of from 2-50 residues chain length. A second advantage of this type of reagent, other than precise structural definition, is the ability to place single or any desired number of chemical handles at certain points in the chain. These can be later used for attachment of recognition haptens and therapeutic drugs at chosen levels of each moiety. For instance, a preferred agent is a 40-mer of 38D-glutamic acid residues, containing two lysene units, the latter of which are epsilon-substituted with recognition units such as DTPA. The remaining glutamic acid residues are then partially substituted with a chemotherapy drug such as taxol, 10-hydroxycamptothecin, 2-pyrrolinodoxorubicin, or melphalan. The remaining glutamic acid residues are then partially substituted with a chemotherapy drug such as taxol, 10-hydroxycamptothecin, 2-pyrrolinodoxorubicin, or melphalan. The substitution ratio is varied, depending on the hydrophobicity of that drug addend. Also, choice of the recognition hapten can also influence overall water solubility of the final conjugate, and for this purpose, hydrophilic moieties such as DTPA are especially preferred. Generally, a substitution ratio of drug to polymer will be in the 10-50% range of available sites, leaving enough unsubstituted residues to maintain the desirable physical properties outlined.

Polymers other than polypeptides can be used within the scope of the invention. Poly(ethylene) glycol [PEG] has desirable in vivo properties for a multi-specific antibody prodrug approach, and can be obtained in a variety of forms having different chemical functionalities at the ends of the polymer. Most PEG derivatives have just two functionally reactive sites, at either end of the polymer chain. Agents derivatized from such PEGs, such as, for example, di-SN-38-PEG can be considered as the shortest member of a class of SN-38-polymer prodrugs. The desirable in vivo properties of PEG derivatives are counter-balanced by the limited loading capacity due to their dimeric functionality. However, more recently, preparation of PEG co-polymers having greater hapten-bearing capacity have been described, such as those described by Poiani et al. (Bioconjugate Chem., 5:62-630, 1994). PEG derivatives activated at both ends, for instance as their bis(succinimidyl) carbonate derivatives are co-polymerized with multi-functional diamines such as lysine. The product of such co-polymerization, containing (-Lys(COOH)-PEG-Lys(COOH)-PEG-)ₙ repeat units wherein the lysyl carboxyl group is not involved in the polymerization process, can be used for attachment of hapten residues such as DTPA or drug residues such as SN-38. The hapten, such as DTPA, or the drug, such as SN-38, may also be reacted with the free carboxyl groups remaining on the termini of the PEG-polylysyl conjugate. Most preferably, a significant amount of amino acid content is used, and the drug will be attached to the amino acid side-chains. The recognition haptens are this appended to the termini of the PEG derivatives.

Other synthetic polymers that can be used to carry recognition haptens and drug include N-(2-hydroxypropyl)methacrylamide (HMPA) copolymers, poly(styrene-co-maleic acid/anhydride (SMA), poly(divinylether maleic anhydride) (DIVEMA), polyethyleneimine, ethoxylated polyethyleneimine, starburst dendrimers and poly(N-vinylpyrrolidone) (PVP). As an example, DIVEMA polymer comprised of multiple anhydride units is reacted with a limited amount of SN-38 to produce a desired substitution ratio of drug on the polymer backbone. Remaining anhydride groups are opened under aqueous conditions to produce free carboxylate groups. A limited number of the free carboxylate groups are activated using standard water-soluble peptide coupling agents (e.g. EDAC (1-ethyl-3-[3-dimethylaminopropyl]carbodiimide)) and coupled to a recognition moiety bearing a free amino group. An example of the latter would be histaminyl-succinyl-glycyl-lysine amide, (HSGK-NH₂) since antibodies have already been raised to the HSG portion of the compound. The free epsilon lysine residue then becomes the point of attachment to the polymer backbone for the recognition hapten. Finally, in certain instances, the polymer used can be a naturally occurring polymer. An instance of this is the use of apo-metallothionein, which is a low MW protein having seven free thiol groups. This protein can be coupled to calicheamicin by disulfide exchange, to produce a disulfide-linked poly-calicheamicin conjugate. Further, the protein can have a limited number of lysyl-residues modified to carry a recognition hapten such as DTPA, DOTA, HSG, etc., prior to any drug conjugation.

Polymers useful for carrying the radionuclide and recognition haptens are selected for their suitability for carrying a particular radionuclide. When the polymer backbone consists of amino-acids, for instance, all of the amino acids of the backbone may be in the D- or L-configuration, or their configurations may be mixed. For example, polymers useful for carrying the radionuclide iodine-131 include a polymer made up of purely D-tyrosine amino acids as well as random co-polymers such as poly(Glu.Tyr) [1:1], poly(Glu.Tyr) [4:1] and poly(Lys.Ala.Glu.Tyr) [5:6:2:1]. In these instances, the polymer contains tyrosine amino acid residues that are readily substituted by radioiodine using methods well known in the art.

In one embodiment, the capture arm of the msAb will recognize a recognition hapten that is appended to the polymer backbone of the polymer conjugate. If the polymer conjugate comprises a separate recognition hapten, the general formula of the polymer conjugate is (recognition hapten)ₙ-(polymer backbone)-(agent)ₘ, where n and m are integers, including zero. However, both n and m can not be zero. If m is zero, then the recognition hapten will necessarily act as the agent that is to be targeted towards the target site. Likewise, if n is zero, then the polymer backbone or the agent will serve as the hapten or antigen for the capture arm.

Examples of recognition haptens include, but are not limited to, metal ion chelates of diethylenetriaminepentaacetic acid (DTPA). Antibodies have been against indium-DTPA by at least two independent laboratories. When used for radioimmunodiagnosis and radioimmunotherapy as low molecular weight complexes, such metal chelates have the desirable property of being rapidly eliminated via the urinary system if they do not bind to a tumor-pretargeted msAb. In this type of recognition system, the antibodies can be reactive against the free chelator or the chelate (metal complex of the chelator). Furthermore, antibodies against indium-DTPA can and do have different affinities for DTPA when the chelator is complexed with different metals. This can be used to an advantage since the capture arm of a multi-specific antibody can be tailored for affinity by simply varying the metal held by the chelate. Antibodies can also be raised against other metal chelators, such as 1,4,7,10-tetrazacyclododecane-N,N′,N″,N″-tetraacetic acid (DOTA), or N, N′ di[2-hydroxy-5-(ethylene-∃-carboxy)benzyl]ethylenediamine N,N′-diacetic acid (HBED). Antibodies raised against the macrocyclic chelate DOTA can be more accepting of different metal substitutions, since the central ring is sterically rigid. Thus, anti-DOTA mAbs may be used with a variety of metal substituents, or even with no metal present at all. In a preferred embodiment, the msAb of the current invention comprises a capture arm that recognizes or binds to DOTA or a metal complex of DOTA.

The recognition hapten of the current invention does not need to be a chelator or a metal chelate. Other low molecular weight molecules capable of generating a strong immune response can be used to prepare antibodies. An instance of this is the histaminyl-succinyl glycine (HSG) hapten, which is a hydrophilic species to which antibodies have been raised. One particular antibody, termed 679, has been described extensively in the scientific literature. This and the anti-chelate type of capture arm of the multi-specific antibody were designed to be hydrophilic in nature, and to be used with low molecular weight diagnostic and therapeutic agents. With the current invention, this aforementioned need for great hydrophilicity is not so stringent, since the polymer will be imparting the bulk of the physical properties of the final therapeutic agent. In turn, this allows for the contemplation of many more immunogens that could be used to raise antibodies useful under in vivo conditions. Commonly used immunogens include fluorescein, 2,4-dinitrophenyl derivatives and others. Additionally, recognition haptens can include amino acid residues contained within a peptide or other molecule.

Alternatively, epitopes comprised within the polymer backbone itself can be used as recognition hapten.

A polymer substituted with a drug of choice can also be used as an immunogen, as can a drug of choice attached to a well-known immunogenic agent such as KLH. The polymer or the drug-polymer conjugate can be attached to a macromolecule to enhance immunogenicity, and that conjugate used as an immunogen, with screening for antibody expression done using standard methods. Production of antibodies against a particular drug can have its advantages, while the production of antibodies against the polymer backbone can have the advantage of producing a ‘universal’ recognition MAb. Thus, as when using distinct recognition units such as DTPA, HSG or DOTA, secondary antibody recognition is not tied to any particular drug, and the same msAb can be used against a variety of drugs conjugated to the same polymer backbone. This embodiment will be useful if two different polymer-drug conjugates will be used in combination (in order to gain the advantage of using several drugs with different modes of action), in a situation that parallels current combination chemotherapy.

The recognition haptens of the polymer conjugate can comprise a known immunogenic recognition moiety, for example, a known hapten. Using a known hapten, for example, fluorescein isothiocyanate (FITC), higher specificity of the polymer conjugate for the antibody is exhibited. This occurs because antibodies raised to the hapten are known and can be incorporated into the inventive multi-specific antibody. Thus, binding of the polymer conjugate with an attached chelator or chelate would be highly specific for the inventive antibody or antibody fragment. Another example of a hapten to be attached onto the polymer conjugate is vitamin B12. The use of vitamin B12 is advantageous since anti-B12 mAbs are known and no free serum B12 exists. Therefore, great specificity for the antibody may be exhibited.

In another embodiment, a radionuclide, used for imaging and/or therapy, may be integrated into the design of the original recognition hapten. For instance, Ac-Gly-D-iodo-Tyr-D-Trp-Gly-D-Lys(Ac)-Gly-D-iodo-Tyr-D-Trp-OH may be used as an immunogen with the express purpose of raising an antibody which is reactive with an iodine-containing peptide, but not with the non-iodo version of the same peptide, namely Ac-Gly-D-Tyr-D-Trp-Gly-D-Lys(Ac)-Gly-D-Tyr-D-Trp-OH. Specificity of antibodies (Abs) for the former over the latter can be demonstrated using standard screening techniques. Of particular importance within this embodiment is the use of astatine-substituted peptides as immunogens to generate Abs and thus msAbs which recognize peptides substituted with alphaparticle-emitting astatine nuclides for radioimmunotherapy (RAIT). In other embodiments, any halogen can be integrated into the design of the original immunogen, including, for example, fluorine-18, bromine, and nuclides of iodine, for example, iodine-124 and iodine-123. Similarly, other non metals can be used, for example ³²P, ³³P and ³⁵S.

As with polymers bearing drug haptens, the msAb used in the invention may be raised against the polymer backbone, the radionuclide-containing hapten, or a hapten separate from the radionuclidic moiety. The latter embodiment is particularly preferred since a discrete number of recognition moieties, for example just one or two, may be added to the polymer independent of the number of radionuclide-haptens that are appended. Antibodies have been raised against specific radionuclide haptens such as indium-DTPA and DOTA, as well as other recognition haptens. One notable example is the antibody termed 679, which was raised against the low MW hapten HSG.

Any useful nuclide may be used within the scope of the invention. Particularly preferred are radionuclides that have useful diagnostic or therapeutic properties, such as indium-11 or yttrium-90, respectively. Other useful nuclides include, but are not limited to, F-18, P-32, Sc-47, Cu-62, Cu-64, Cu-67, Ga-67, Ga-68, Y-86, Y-90, Zr-89, Tc-99m, Pd-109, Ag-111, In-111, I-123, I-125, I-131, Sm-153, Gd-155, Gd-157, Tb-161, Lu-177, Re-186, Re-188, Pt-197, Pb-212, Bi-212, Bi-213, Ra-223, Ac-225, As-72, As-77, At-211, Au-198, Au-199, Bi-212, Br-75, Br-76B, C-11, Co-55Co, Dy-166, Er-169, F-18, Fe-52, Fe-59, Ga-67, Ga-68, Gd-154-158, Ho-166, I-120, I-121, I-124, In-110, In-111, Iri194, Lu-177, Mn-51, Mn-52, Mo-99, N-13, O-15, P-32, P-33, Pb-211, Pb-212, Pd-109, Pm-149, Pr-142, Pr-143, Rb-82, Re-189, Rh-105, Sc-47, Se-75, Sr-83, Sr-89, Tb-161, Tc-94, Tc-99, Y-86, Y-90 or Zr-89.

For example, suitable diagnostic radionuclides include In-110, In-111, Lu-177, F-18, Fe-52, Cu-62, Cu-64, Cu-67, Ga-67, Ga-68, Y-86, Y-90, Zr-89, Tc-94m, Tc-94, Tc-99m, I-120, I-123, I-124, I-125, I-131, Gd-154-158, P-32, C-11, N-13, O-15, Re-186, Re-188, Mn-51, Mn-52m, Co-55, As-72, Br-75, Br-76, Rb-82m and Sr-83. A typical diagnostic radionuclide emits particles and/or positrons having between 25-10,000 keV.

Additionally, suitable therapeutic radionuclides include, but are not limited to In-111, Lu-177, Bi-212, Bi-213, At-211, Cu-62, Cu-64, Cu-67, Y-90, 1-125, I-131, P-32, P-33, Sc-47, Ag, 67Ga-111, Pr-142, Sm-153, Tb-161, Dy-166, Ho-166, Re-186, Re-188, Re-189, Pb-212, Ra-223, Ac-225, Fe-59, Se-75, As-77, Sr-89, Mo-99, Rh-105, Pd-109, Pr-143, Pm-149, Er-169, Ir-194, Au-198, Au-199, Ac-225 and Pb-211. A typical therapeutic cation emits particles and/or positrons having between 20-10,000 keV.

Tight binding of radiometallic nuclides often requires a chelating agent for the radiometal. Naturally-occurring polymeric chelating agents, such as the protein apo-metallothionein, can be used. Standard radiolabeling methods and precautions used in the radiolabeling of low molecular weight chelates may be used to prepare radiolabeled chelate polymers. For instance, procedures using radiometals, such as indium-111 and yttrium-90, generally require highly pure supplies of the radionuclide, deionized water in all buffer solutions, and acid-washing of glassware and plastic-ware used with any of the reagents during the radiolabeling procedures. Procedures using radiometals such as rhenium-188, which require a chemical reduction step to effect labeling, are best carried out using oxygen-depleted buffers and argon atmospheres overlaying the radiolabeling reactions.

The polymer conjugate may be conjugated to a variety of agents useful for treating or identifying diseased tissue. Preferably the agents that are conjugated to the polymer conjugate are selected from the group consisting of therapeutic agents, peptides, enzymes and labeled ligands. As used herein, the term therapeutic agent is used to mean any compound or molecule that will either cause, elicit or initiate a cellular or physiological response within the targeted tissue. Examples of cellular or physiological responses, which should be obvious to one skilled in the art, include, but are not limited to ion influx or efflux, initiation of second messenger pathways, synthesis of DNA, translation of mRNA, entry of the cells into the cell cycle, arrest of the cell in the cell cycle, endocytosis, release of molecules from the cell, exocytosis, cytosolic proteins acting on an internalized ligand, non-programmed cell death (cellular toxicity) and apoptosis. Examples of therapeutic agents for use with the invention include, but are not limited to, metal chelate complexes, drugs, prodrugs, radionuclides, boron addends, labeling compounds, toxins and other effector molecules, such as cytokines, lymphokines, chemokines, immunomodulators, radiosensitizers, asparaginase, boron addends and radioactive halogens. Preferably, the therapeutic agent that is conjugated to the polymer backbone is selected from the group consisting of therapeutic radioisotpes, toxins, drugs, prodrugs and boron addends. Suitable toxins include ricin, abrin, ribonuclease, DNase I, Staphylococcal enterotoxin-A, pokeweed antiviral protein, gelonin, diphtherin toxin, Pseudomonas exotoxin, or Pseudomonas endotoxin.

A cytokine, for the purposes of this disclosure, include all known cytokines including, at least, IL-1, IL-2, IL-3, IL-6, IL-10, IL-12, IL-18, IL-21, interferon-α, interferon-β, and interferon-γ. It may also be a colony stimulating factor, such as GM-CSF, G-CSF, erythropoietin, thrombopoietin, and the like.

As used herein, the term agent, ligand or compound is intended to mean a protein, nucleic acid, carbohydrate, lipid, a polymer or a small molecule.

Drugs for use with the current invention include, but are not limited to, any currently approved or not-yet-approved chemotherapy drug, as long as it can be attached to the polymer conjugate. Typically useful drugs include, but are not limited to, the following agents and derivatives of these agents: at least, the taxanes, nitrogen mustards, ethylenimine derivatives, alkyl sulfonates, nitrosoureas, triazenes; folic acid analogs, pyrimidine analogs, purine analogs, vinca alkaloids, antibiotics, enzymes, platinum coordination complexes, substituted urea, methyl hydrazine derivatives, adrenocortical suppressants, or antagonists. More specifically, the chemotherapeutic agents may be steroids, progestins, estrogens, antiestrogens, or androgens. Even more specifically, the chemotherapy agents may be azaribine, anastrozole, azacytidine, bleomycin, bryostatin-1, busulfan, carmustine, chlorambucil, cisplatin, irinotecan (CPT-11), carboplatin, cladribine, celebrex, cyclophosphamide, cytarabine, dacarbazine, docetaxel, dactinomycin, daunorubicin, dexamethasone, diethylstilbestrol, doxorubicin, ethinyl estradiol, estramustine, etoposide, floxuridine, fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, methotrexate, mitoxantrone, mithramycin, mitomycin, mitotane, phenyl butyrate, prednisone, procarbazine, paclitaxel, pentostatin, semustine streptozocin, tamoxifen, taxanes, taxol, testosterone propionate, thalidomide, thioguanine, thiotepa, teniposide, topotecan, uracil mustard, vinblastine, vinorelbine or vincristine.

Additionally, the polymer conjugate may comprise a therapeutic agent consisting of boron addends to be used in Boron Neutron Capture Therapy (BNCT) protocols. BNCT is a binary system designed to deliver ionizing radiation to tumor cells by neutron irradiation of tumor-localized boron-10 atoms. BNCT is based on the nuclear reaction which occurs when a stable isotope, isotopically enriched B-10 (present in 19.8% natural abundance), is irradiated with thermal neutrons to produce an alpha particle and a Li-7 nucleus. These particles have a path length of about one cell diameter, resulting in high linear energy transfer. Just a few of the short-range 1.7 MeV alpha particles produced in this nuclear reaction are sufficient to target the cell nucleus and destroy it. Success with BNCT of cancer requires methods for localizing a high concentration of boron-10 at tumor sites, while leaving non-target organs essentially boron-free. Compositions and methods for treating tumors in patients using pre-targeting msAb for BNCT are described in U.S. Pat. No. 6,228,362 and can easily be modified in accordance with the present invention, and is hereby incorporated by reference. Additionally, other elements are suitable for neutron capture reactions. One example is uranium. Uranium, in large amounts, can be bound by naturally occurring chelating agents such as ferritin. Such strategies have been described in the art, for example U.S. Pat. No. 6,228,362 and references cited therein, are easily adaptable to the present invention and are hereby incorporated in their entirety by reference.

Additionally, peptides and enzymes may be conjugated to polymer conjugate. Enzymes and peptides conjugated to the polymer conjugate may be useful for such things as activating a prodrug, improving the efficacy of a normal therapeutic agent by controlling the body's detoxification pathways, acting as a co-factor, acting as a ligand for other proteins or increasing the target-specific toxicity of a drug.

In one embodiment of the current invention, a msAb is first administered to the subject, followed by administration of a polymer-enzyme conjugate. After the enzyme is pre-targeted to the target site, a cytotoxic drug is injected, which is known to act at the target site, or a prodrug form thereof which is converted to the drug in situ by the pre-targeted enzyme. The drug is one which is detoxified to form an intermediate of lower toxicity, most commonly a glucuronide, using the mammal's ordinary detoxification processes. The detoxified intermediate, e.g., the glucuronide, is reconverted to its more toxic form by the pre-targeted enzyme and thus has enhanced cytotoxicity at the target site. This results in a recycling of the drug. Similarly, an administered prodrug can be converted to an active drug through normal biological processes. The pre-targeted enzyme improves the efficacy of the treatment by recycling the detoxified drug. This approach can be adopted for use with any enzyme-drug pair. Similar pre-targeting strategies have been described in U.S. Ser. No. 09/399,021. Those methodologies are easily adaptable to the present invention and are hereby incorporated in their entirety by reference.

In an alternative embodiment, the enzyme-polymer conjugate can be mixed with the targeting msAb prior to administration to the subject. After a sufficient time has passed for the enzyme-polymer-msAb conjugate to localize to the target site and for unbound conjugate to clear from circulation, a prodrug is administered. As discussed above, the prodrug is then converted to the drug in situ by the pre-targeted enzyme.

As used herein, the term prodrug is used to mean a therapeutic agent that is administered in an inactive state and is subsequently converted to a more active state. Additionally, prodrug is also used to mean an agent that is active upon administration, and is subsequently converted to a more active state. Furthermore, prodrug may also mean an agent that is not specific in its activity upon administration, and is subsequently converted to a more specific-acting agent. As described above, the conversion of a prodrug may take place either within the subject or not. The conversion may also be a natural process where the prodrug is naturally metabolized by the body to a more active or specific agent, or it may be a synthetic process where an additional agent is administered to convert the prodrug to a more active or specific state.

Certain cytotoxic drugs that are useful for anticancer therapy are relatively insoluble in serum. Some are also quite toxic in an unconjugated form, and their toxicity is considerably reduced by conversion to prodrugs. Conversion of a poorly soluble drug to a more soluble conjugate, e.g., a glucuronide, an ester of a hydrophilic acid or an amide of a hydrophilic amine, will improve its solubility in the aqueous phase of serum and its ability to pass through venous, arterial or capillary cell walls and to reach the interstitial fluid bathing the tumor. Cleavage of the prodrug deposits the less soluble drug at the target site. Many examples of such prodrug-to-drug conversions are disclosed in Hansen U.S. Pat. No. 5,851,527.

Conversion of certain toxic substances such as aromatic or alicyclic alcohols, thiols, phenols and amines to glucuronides in the liver is the body's method of detoxifying them and making them more easily excreted in the urine. One type of anti-tumor drug that can be converted to such a substrate is epirubicin, a 4-epimer of doxorubicin (Adriamycin), which is an anthracycline glycoside and has been shown to be a substrate for human beta-D-glucuronidase See, e.g., Arcamone, Cancer Res., 45:5995, 1985. Other analogues with fewer polar groups are expected to be more lipophilic and show greater promise for such an approach. Other drugs or toxins with aromatic or alicyclic alcohol, thiol or amine groups are candidates for such conjugate formation. These drugs, or other prodrug forms thereof, are suitable candidates for the site-specific enhancement methods of the present invention.

The prodrug CPT-11 (irinotecan) is converted in vivo by carboxylesterase to the active metabolite SN-38. Although SN-38 is a highly effective anti-tumor agent, therapeutic doses can not be administered to subjects due to its toxicity. One application of the invention, therefore, is to target such therapies to the tumor site using a msAb specific for a tumor-associated antigen and a hapten (e.g. di-DTPA) followed by injection of a di-DTPA-carboxylesterase-polymer conjugate. Once a suitable tumor-to-background localization ratio has been achieved, the CPT-11 is given and the tumor-localized carboxylesterase serves to convert CPT-11 to SN-38 at the tumor. Due to its poor solubility, the active SN-38 will remain in the vicinity of the tumor and, consequently, will exert an effect on adjacent tumor cells that are negative for the antigen being targeted. This is a further advantage of the method. Modified forms of carboxylesterases have been described and are within the scope of the invention. See, e.g., Potter et al., Cancer Res., 58:2646-2651 and 3627-3632, 1998.

Etoposide is a widely used cancer drug that is detoxified to a major extent by formation of its glucuronide and is within the scope of the invention. See, e.g., Hande et al., Cancer Res., 48: 1829-1834, 1988. Glucuronide conjugates can be prepared from cytotoxic drugs and can be injected as therapeutics for tumors pre-targeted with mAb-glucuronidase conjugates. See, e.g., Wang et al., Cancer Res., 52:4484-4491, 1992. Accordingly, such conjugates also can be used with the pre-targeting approach described here. Similarly, designed prodrugs based on derivatives of daunomycin and doxorubicin have been described for use with carboxylesterases and glucuronidases. See, e.g., Bakina et al., J. Med Chem., 40:4013-4018, 1997. Other examples of prodrug/enzyme pairs that can be used within the present invention include, but are not limited to, glucuronide prodrugs of hydroxy derivatives of phenol mustards and beta-glucuronidase; phenol mustards or CPT-11 and carboxypeptidase; methotrexate-substituted alpha-amino acids and carboxypeptidase A; penicillin or cephalosporin conjugates of drugs such as 6-mercaptopurine and doxorubicin and beta-lactamase; etoposide phosphate and alkaline phosphatase.

In other embodiments of the present invention, the enzyme capable of activating a prodrug at the target site or improving the efficacy of a normal therapeutic by controlling the body's detoxification pathways is conjugated to the recognition hapten. The enzyme-hapten-polymer conjugate is administered to the subject following administration of the pre-targeting msAb and is directed to the target site. After the enzyme is localized at the target site, a cytotoxic drug is injected, which is known to act at the target site, or a prodrug form thereof which is converted to the drug in situ by the pre-targeted enzyme. As discussed above, the drug is one which is detoxified to form an intermediate of lower toxicity, most commonly a glucuronide, using the mammal's ordinary detoxification processes. The detoxified intermediate, e.g., the glucuronide, is reconverted to its more toxic form by the pre-targeted enzyme and thus has enhanced cytotoxicity at the target site. This results in a recycling of the drug. Similarly, an administered prodrug can be converted to an active drug through normal biological processes. The pretargeted enzyme improves the efficacy of the treatment by recycling the detoxified drug. This approach can be adopted for use with any enzyme-drug pair. In an alternative embodiment, the enzyme-hapten-polymer conjugate can be mixed with the targeting msAb prior to administration to the subject. After a sufficient time has passed for the enzyme-hapten-polymer-msAb conjugate to localize to the target site and for unbound conjugate to clear from circulation, a prodrug is administered. As discussed above, the prodrug is then converted to the drug in situ by the pre-targeted enzyme.

In another embodiment of the present invention, the polymer conjugate may be conjugated to a prodrug. The pre-targeting msAb is administered to the subject and allowed to localize to the target and substantially clear circulation. At an appropriate later time, a polymer conjugate comprising a prodrug, for example poly-glutamic acid (SN-38-ester)₁₀, is given, thereby localizing the prodrug specifically at the tumor target. It is known that tumors have increased amounts of enzymes released from intracellular sources due to the high rate of lysis of cells within and around tumors. A practitioner can capitalize on this fact by appropriately selecting prodrugs capable of being activated by these enzymes. For example, carboxylesterase activates the prodrug poly-glutamic acid (SN-38-ester)₁₀ by cleaving the ester bond of the poly-glutamic acid (SN-38-ester)₁₀ releasing large concentrations of free SN-38 at the tumor. Alternatively, the appropriate enzyme also can be targeted to the tumor site.

After cleavage from the polymer conjugate, the drug is internalized by the tumor cells. Alternatively, the drug can be internalized as part of an intact complex by virtue of cross-linking at the target. The polymer conjugate may induce internalization of tumor-bound msAb and thereby improve the efficacy of the treatment by causing higher levels of the drug to be internalized.

A variety of prodrugs can be conjugated to the polymer conjugate. The above exemplifications of polymer use are concerned with SN-38, the active metabolite of the prodrug CPT-11 (irinotecan). SN-38 has an aromatic hydroxyl group that was used in the above descriptions to produce aryl esters susceptible to esterase-type enzymes. Similarly the camptothecin analog topotecan, widely used in chemotherapy, has an available aromatic hydroxyl residue that can be used in a similar manner as described for SN-38, producing esterase-susceptible polymer-prodrugs.

Doxorubicin also contains aromatic hydroxyl groups that can be coupled to carboxylate-containing polymeric conjugates using acid-catalyzed reactions similar to those described for the camptothecin family. Similarly, doxorubicin analogs like daunomycin, epirubicin and idarubicin can be coupled in the same manner. Doxorubicin and other drugs with amino ‘chemical handles’ active enough for chemical coupling to polymeric conjugates can be effectively coupled to conjugates via these free amino groups in a number of ways. Polymers bearing free carboxylate groups can be activated in situ and the activated polymers mixed with doxorubicin to directly attach the drug to the side-chains of the polymer via amide bonds. Amino-containing drugs can also be coupled to amino-pendant polymers by mixing commercially available and cleavable cross-linking agents, such as ethylene glycobis(succinimidylsuccinate) (EGS) (Pierce Chemical Co., Rockford, Ill.) or bis-[2-(succinimido-oxycarbonyloxy)ethyl]sulfone (BSOCOES) (Molecular Biosciences, Huntsville, Ala.), to cross-link the two amines as two amides after reaction with the bis(succinimidyl) ester groups. This is advantageous as these groups remain susceptible to enzymatic cleavage. For example, (doxorubicin-EGS)ₙ-poly-lysine remains susceptible to enzymatic cleavage of the diester groups in the EGS linking chain by enzymes such as esterases. Doxorubicin also can be conjugated to a variety of peptides, for example, HyBnK(DTPA)YK(DTPA)-NH₂, using established procedures (HyBn=p-H₂NNHC₆H₄CO₂H). See Kaneko et al., J. Bioconjugate Chem., 2: 133-141, 1991.

In one preferred embodiment, the therapeutic agent conjugated to the polymer conjugate comprises doxorubicin coupled to the polymer conjugate comprising amine residues and a chelating agent, such as DTPA, to form a DTPA-polymeric peptide-doxorubicin conjugate, wherein the DTPA forms the recognition hapten for a pretargeted bsMAb. Preferably, the polymer conjugate comprises a tyrosyl-lysine dipeptide, e.g., poly[Tyr-Lys](DTPA)-NH₂, and more preferably still it comprises poly[Lys(DTPA)-Tyr-Lys(DTPA)]-NH₂. Doxorubicin phenyl hydrazone conjugates to bis-DPTA containing peptides are particularly desirable in a therapeutic context.

Methotrexate also has an available amino group for coupling to activated carboxylate-containing polymers, in a similar manner to that described for doxorubicin. It also has two glutamyl carboxyl groups (alpha and gamma) that can be activated for coupling to amino-group containing polymers. The free carboxylate groups of methotrexate can be activated in situ and the activated drug mixed with an amino-containing polymer to directly attach the drug to the side-chains of the polymer via amide bonds. Excess unreacted or cross-reacted drug is separated readily from the polymer-drug conjugate using size-exclusion or ion-exchange chromatography.

Maytansinoids and calicheamicins (such as esperamycin) contain mixed di- and tri-sulfide bonds that can be cleaved to generate species with a single thiol useful for chemical manipulation. The thiomaytansinoid or thioespera-mycin is first reacted with a cross-linking agent such as a maleimido-peptide that is susceptible to cleavage by peptidases. The C-terminus of the peptide is then activated and coupled to an amino-containing polymer such as polylysine.

In still other embodiments, the multi-specific antibody-directed delivery of therapeutics or prodrug polymers to in vivo targets can be combined with multi-specific antibody delivery of radionuclides, such that combination chemotherapy and radioimmunotherapy is achieved. Each therapy can be conjugated to the polymer conjugate and administered simultaneously, or the nuclide can be given as part of a first polymer conjugate and the drug given in a later step as part of a second polymer conjugate. In one simple embodiment, a polymer containing a single prodrug and a single nuclide is constructed. For example, a polymer conjugate, where the polymer backbone is composed of a peptide polymer, can be used, whereby SN-38 is attached to the gamma glutamyl carboxyl group as an aryl ester, while the chelate DOTA is attached to the epsilon amino group as an amide, to produce a polymer-prodrug-recognition hapten complex, for example poly[Glu(SN-38)₁₀-Lys(Y-90-DOTA)₂]. The DOTA chelate can then be radiolabeled with various metals for imaging and therapy purposes including In-111, Y-90, Sm-153, Lu-177 and Zr-89. As the metal-DOTA complex may represent the recognition hapten on the polymer conjugate, the only requirement for the metal used as part of the DOTA complex is that the secondary recognition antibody also used recognizes that particular metal-DOTA complex at a sufficiently high affinity. Generally, this affinity (log Kₐ) is between 6-11. Also, triply substituted polymers can be used, such as poly[Glu(Sn-38)₁₀-Lys(Y-90-DOTA)ₙ(histamine-succinate)ₘ, where n and m are integers, such that the recognition hapten is independent of the radioimmunotherapy (therapeutic) agent. The prodrug is then activated by carboxylesterases present at the tumor site or by carboxylesterases targeted to the site using a second polymer conjugate.

Alternatively, a combination therapy can be achieved by administering the chemotherapy and radioimmunotherapy agents in separate steps. For example, a subject expressing CEA-tumors is first administered msAb with at least one arm which specifically binds CEA and at least one other arm which specifically binds the polymer whose recognition hapten is a conjugate of yttrium-DOTA. Later the subject is treated with a polymer conjugate comprising a conjugate of yttrium-DOTA-beta-glucuronidase. After sufficient time for msAb and enzyme localization and clearance, a second polymer conjugate is given. The second polymer conjugate localizes to the tumor by virtue of msAb at the tumor that are not already bound to a first polymer conjugate. Localization of both the prodrug and its respective enzyme to the target site enhances the production of active drug by ensuring that the enzyme is not substrate limited. This embodiment constitutes a marked improvement of current prodrug methodologies currently practiced in the art.

Another advantage of administering the prodrug-polymer in a later step, after the nuclide has been delivered as part of a previously given polymer conjugate, is that the synergistic effects of radiation and drug therapy can be manipulated and, therefore, maximized. It is hypothesized that tumors become more ‘leaky’ after RAIT due to radiation damage. This can allow a polymer-prodrug to enter a tumor more completely and deeply. This results in improved chemotherapy.

Alternatively, the RAIT therapy agent can be attached to msAb rather than the polymer conjugate. For example, an anti-CEA×anti-DTPA msAb conjugated to Y-90-DOTA is administered first to a subject with CEA-expressing tumors. In this instance, advantage is taken of the selectivity of certain anti-chelate mAbs in that an anti-indium-DTPA antibody does not bind to a yttrium-DOTA chelate. After the Y-90-DOTA-anti-CEA×anti-indium-DTPA has maximized at the tumor and substantially cleared non-target tissue, a polymer conjugate comprising indium-DTPA-glucuronidase is injected and localized specifically to the CEA tumor sites. The subject is then injected with a polymer-prodrug such asipoly(Glu)(SN-38)₁₀. The latter is cleaved selectively at the tumor to active monomeric SN-38, successfully combining chemotherapy with the previously administered RAIT.

It should also be noted that a multi-specific antibody or antibody fragment can be used in the present method, with at least one binding site specific to an antigen at a target site and at least one other binding site specific to an enzyme. Such an antibody can bind the enzyme prior to injection, thereby obviating the need to covalently conjugate the enzyme to the antibody, or it can be injected and localized at the target site and, after non-targeted antibody has substantially cleared from the circulatory system of the subject, the enzyme can be injected in an amount and by a route which enables a sufficient amount of the enzyme to reach the pre-targeted msAb and bind to it to form an antibody-enzyme conjugate in situ.

The polymer conjugate may also be conjugated to a variety of agents that act as labeling ligands that can be useful for identifying normal or diseased tissue. In one preferred embodiment of the current invention, the agents that are conjugated to the polymer conjugate are labeled ligands, also referred to as diagnostic agents. Examples of labeled ligands include, but are no limited to, radioisotopes, coloring agents (such as the biotin-streptavidin complex), contrasting agents, fluorescent compounds or molecules and enhancing agents for magnetic resonance imaging (MRI). Preferably, the labeled ligands are selected from the group consisting of radioisotopes, enhancing agents for use in magnetic resonance imaging, contrasting agents and coloring agents.

In the practice of one embodiment of the invention, the msAb is administered prior to administration of a diagnostic agent which is conjugated with the polymer conjugate. After sufficient time has passed for the msAb to target to the diseased tissue, the diagnostic agent is administered. Subsequent to administration of the diagnostic agent, imaging can be performed. Tumors can be detected in body cavities by means of directly or indirectly viewing various structures to which light is delivered and then collected. Lesions at any body site can be viewed so long as nonionizing radiation can be delivered and recaptured from these structures. For example, positron emission tomography (PET) which is a high resolution, non-invasive, imaging technique can be used with the inventive antibodies for the visualization of human disease. In PET, 511 keV gamma photons produced during positron annihilation decay are detected. Similar pre-targeting strategies for PET using Fluorine-18 and Gallium-68 have been described, respectively in U.S. Pat. No. 6,187,284 and U.S. Ser. No. 09/644,706. The methodologies described in these applications are easily adaptable to the present invention and are hereby incorporated in their entirety by reference.

The present invention also contemplates intravascular and endoscopic methods for diagnosing/detecting a disease or disorder comprising: (a) administering to a tissue a multi-specific antibody or antibody fragment, comprising a targeting arm that binds to an antigen on said target site, and a capture arm that binds to a polymer conjugate; and (b) administering to said tissue a polymer conjugate that binds to said capture arm, said polymer conjugate comprising a polymer conjugated to a diagnostic agent.

A probe can be used in the form of an endoscope, and inserted into a body cavity through an orifice, such as the mouth, nose, ear, anus or vagina. The term endoscope is herein used generically to refer to an anally introduced endoscope, an orally introduced bronchoscope, a urethrally introduced cystoscope, or the like. Certain of these may benefit greatly from further progress in miniaturization of components and their utility to practice the method of the present invention will be enhanced as a function of the development of suitably microminiaturized components for this type of instrumentation. Highly miniaturized probes which could be introduced invascularly, e.g., via catheters or the like, are also suitable for use in the method of the invention.

A tumor, for example, can be detected in a body cavity by means of directly or indirectly viewing a structure to which light is delivered and then collected. A lesion at any body site can be viewed so long as nonionizing radiation can be delivered and recaptured from these structures. Imaging approaches are described in Dougherty et al., Cancer Res. 38:2628, 1978; Dougherty, T. J., Photochem. Photobiol. 45:879, 1987; Jori and Perria, eds., Photodynamic Therapy of Tumors and Other Diseases; Padua: Libreria Progetto, 1985; Profio, Proc. Soc. Photoopt. Instr. Eng. 907:150, 1988; Doiron and Gomer, eds., Porphyrin Localization and Treatment of Tumors; New York: Alan Liss, 1984; Hayata and Dougherty, Lasers and Hematoporphyrin Derivative in Cancer; Tokyo: Igaku-Shoin, 1984; and van den Bergh, Chem. Britain 22:430, 1986, incorporated herein in their entirety by reference.

It is known that dyes can be attached to antibodies for a more specific binding to certain tissues and cells, including malignant and normal cells, depending upon the discriminatory power of the antibodies in question. In cancer, for example, such labeled antibodies have been used in flow cytometry and in immunohistology to stain malignant cells with many different kinds of anticancer antibodies, as described, for example, in Goding, Monoclonal Antibodies: Principles and Practice; London/New York, Academic Press, 1983; Ferrone and Dierich, eds., Handbook of Monoclonal Antibodies; Park Ridge, N.J., Noyes Publications, 1985; Wick and Siegal, eds., Monoclonal Antibodies in Diagnostic Immunohistochemistry; New York/Basel, Marcel Dekker, 1988, incorporated herein in their entirety by reference. Fluorescent and other chromagens, or dyes, such as porphyrins sensitive to visible light, have been used to detect and even treat lesions by directing the suitable light to the tumor or lesion (cited above). In therapy, this has been termed “photoradiation, phototherapy, or photodynamic therapy (Jori and Perria, eds., Photodynamic Therapy of Tumors and Other Diseases, Padua: Libreria Progetto, 1985; van den Bergh, Chem. Britain 22:430, 1986).

Monoclonal antibodies have been coupled with photoactivated dyes for achieving a photodetection or photocopy (Mew et al., J. Immunol. 130:1473, 1983; idem., Cancer Res. 45:4380, 1985; Oseroff et al., Proc. Natl. Acad. Sci. USA 83:8744, 1986; idem., Photochem. Photobiol. 46:83, 1987; Hasan et al., Prog. Clin. Biol. Res. 288:471, 1989; Tatsuta et al., Lasers Surg. Med. 9:422, 1989; Pelegrin et al., Cancer 67:2529, 1991—all incorporated in their entirety herein by reference).

Thus, the present inventive antibodies or antibody fragments can be used in a method of photodynamic diagnosis or therapy. In the diagnostic method, a diagnostic agent is injected, for example, systemically, and laser-induced fluorescence can be used by endoscopes to detect sites of cancer which have accreted the light-activated agent. For example, this has been applied to fluorescence bronchoscopic disclosure of early lung tumors (Doiron et al., Chest 76:32, 1979), incorporated herein by reference. In another example, the inventive antibodies and antibody fragments can be used in single photon emission. For example, a Tc-99m-labeled diagnostic agent can be administered to the subject following administration of the msAbs. The subject is then scanned with a gamma camera which produces single-photon emission computed tomographic images and defines the lesion or tumor site.

The present invention also can be used in a method for photodynamic therapy. In this methodology, a photosensitizer, for example a hematoporphyrin derivative such as dihematoporphyrin ether, is administered to the subject. Anti-tumor activity is initiated by the use of strong red light, for example, at 630 nanometers wavelength. Alternate photosensitizers can be utilized, including those useful at longer wavelengths, where skin is less photosensitized by the sun. Examples of such photosensitizers include, but are not limited to, benzoporphyrin monoacid ring A (BPD-MA), tin etiopurpurin (SnET2), sulfonated aluminum phthalocyanine (AlSPc) and lutetium texaphyrin (Lutex).

The msAb can be given at some time prior to administration of the polymer conjugate. The doses and timing of the reagents can be readily worked out by a skilled artisan, and are dependent on the specific nature of the reagents employed. If a msAb-F(ab′)₂ derivative is given first, then a waiting time of 1-6 days before administration of the polymer conjugate would be appropriate. If an IgG-Fab′msAb conjugate is the primary targeting vector, then a longer waiting period before administration of the polymer conjugate would be indicated, probably in the range of 3-15 days. If a multi-specific fusion protein, for example an anti-CEA Fab×anti-peptide scFv, is the primary targeting vector, a shorter waiting period before administration of the polymer conjugate would be indicated, probably in the range of 1-5 days.

The method of targeting an agent towards a target site also includes administering a clearing composition to the tissue to clear the unbound msAb from the tissue. The clearing agent is given between doses of the msAb and the polymer conjugate. The present inventors have discovered that a clearing agent consisting of a glycosylated anti-idiotypic Fab′ fragment that recognizes or binds to the targeting arms of the msAb. In this embodiment, a msAb is given and allowed to accrete in the targeted site. To clear residual msAb, an anti-idiotypic Ab to the msAb is given as a glycosylated Fab′ fragment. The clearing agent binds to the msAb in a monovalent manner, while its appended glycosyl residues direct the entire complex to the liver, where rapid metabolism takes place. The polymer conjugate, is subsequently given to the subject. For example, an anti-CEA (MN 14 Ab)×anti-peptide msAb is given to the tissue and the msAb is allowed to accrete in the desired targeted site to its maximum extent. To clear residual msAb, an anti-idiotypic Ab to MN-14, termed WI2, is given as a glycosylated Fab′ fragment. The WI2 Ab to the MN-14 arm of the msAb has a high affinity and the clearance mechanism differs from other disclosed mechanisms (see Goodwin, et al., id), as it does not involve cross-linking, because the WI2-Fab′ is a monovalent moiety.

The current invention also provides a kit useful for targeting a target site within a tissue in a subject or tissue sample comprising, (a) a multi-specific antibody or antibody fragment comprising a targeting arm that binds to an antigen within said tissue, and a capture arm that binds to a polymer conjugate; and (b) a polymer conjugate that binds to said capture arm, said polymer conjugate comprising a polymer conjugated to an agent selected from the group consisting of a therapeutic agent, a peptide, an enzyme and a labeled ligand. Instruments which facilitate identifying or treating diseased tissue also may be included in the kit. Examples include, but are not limited to application devices, such as syringes. Solutions required for utilizing the disclosed invention for identifying or treating diseased tissue also may be included in the kit.

In a preferred embodiment, the polymer conjugate of the kit, as provided by the current invention, further comprises a recognition hapten.

In a preferred embodiment, the msAb of the kit may be monoclonal or polyclonal in nature, but preferably monoclonal. Furthermore, the targeting arm and the capture arm of the msAb may be monoclonal or polyclonal in nature. Preferably, either the target arm or the capture arm is monoclonal. Most preferably, the target arm and the capture arm are both monoclonal.

In another preferred embodiment, the msAb of the kit may be engineered to possess a label. Examples of labels that the msAb may possess include, but are not limited to, a labeling ligand such as the biotin-streptavidin complex and radioisotopes. Preferably, the msAb of the current invention is radiolabeled.

In another preferred embodiment, the msAb of the kit may be chimeric humanized or human, but more preferably human or humanized. In still another preferred embodiment, the targeting arm and the capture arm of the msAb may be chimeric human or humanized. Preferably, either the target arm or the capture arm is human. Most preferably, the target arm and the capture arm are both human or humanized.

In one preferred embodiment, the kit as provided by the current application may also include a clearing composition that will clear the unbound msAb from the tissue. The clearing agent is preferably given between administering or applying the msAb and administration or application of the polymer conjugate.

In another preferred embodiment, the kit of the current invention may also include a drug or prodrug. In a more preferred embodiment, the kit contains a polymer conjugate that is conjugated to an enzyme, which will convert the prodrug to an active drug.

The invention of the current application, in general, relates to a msAb/polymer conjugate recognition system for targeting tissues for disease treatment or diagnosis. Accordingly, contemplated in the present invention is a method for diagnosing or treating a disease or disorder in a patient. For example, the methods of the instant invention can be used to diagnose or treat malignancies, inflammatory diseases, including autoimmune diseases, and infectious diseases, such as a bacterial, fungal, parasitic or viral lesion. Infectious diseases include those caused by invading microbes or parasites. As used herein, “microbe” denotes virus, bacteria, rickettsia, mycoplasma, protozoa, fungi and like microorganisms, “parasite” denotes infectious, generally microscopic or very small multicellular invertebrates, or ova or juvenile forms thereof, which are susceptible to antibody-induced clearance or lytic or phagocytic destruction, e.g., malarial parasites, spirochetes and the like, including helminths, while “infectious agent” or “pathogen” denotes both microbes and parasites.

For example, the fungus may be from the species of Microsporum, Trichophyton, Epidermophyton, Ssporothrix schenckii, Cyrptococcus neoformans, Coccidioides immitis, Histoplasma capsulatum, Blastomyces dermatitidis, or Candida albicans; the virus may be from the species of human immunodeficiency virus (HIV), herpes virus, cytomegalovirus, rabies virus, influenza virus, hepatitis B virus, Sendai virus, feline leukemia virus, Reo virus, polio virus, human serum parvo-like virus, simian virus 40, respiratory syncytial virus, mouse mammary tumor virus, Varicella-Zoster virus, Dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus and blue tongue virus; the bacterium may be, for example, Anthrax bacillus, Streptococcus agalactiae, Legionella pneumophilia, Streptococcus pyogenes, Escherichia coli, Neisseria gonorrhoeae, Neisseria meningitidis, Pneumococcus, Hemophilis influenzae B, Treponema pallidum, Lyme disease spirochetes, Pseudomonas aeruginosa, Mycobacterium leprae, Brucella abortus, Mycobacterium tuberculosis and Tetanus toxin; the parasite may be a helminth or a malarial parasite; the protozoan may be Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japanicum, Babesia bovis, Elmeria tenella, Onchocerca volvulus, Leishmania tropica, Trichinella spiralis, Onchocerca volvulus, Theileria parva, Taenia hydatigena, Taenia ovis, Taenia saginata, Echinococcus granulosus or Mesocestoides corti; and the mycoplasma may be Mycoplasma arthritidis, Mycoplasma hyorhinis, Mycoplasma orale, Mycoplasma arginini, Acholeplasma laidlawii, Mycoplasma salivarum, and Mycoplasma pneumoniae. Listings of representative disease-causing infectious organisms to which antibodies can be developed for use in this invention are contained in the second and subsequent editions of Davis et al, “Microbiology” (Harper & Row, New York, 1973 and later), and are well known to one of ordinary skill in the art.

Other diseases or disorders include a solid tumor, a B-cell malignancy and a T-cell malignancy. Preferably, the B-cell malignancy is selected from the group consisting of indolent forms of B-cell lymphomas, aggressive forms of B-cell lymphomas, chronic lymphatic leukemias, multiple myeloma, and acute lymphatic leukemias. Also preferred, the solid tumor is selected from the group consisting of a melanoma, sarcoma, glioma and carcinoma, including a renal carcinoma, lung carcinoma, intestinal carcinoma, and stomach carcinoma. The polymer conjugate, and its chemistry, are vital to the success of the current invention.

With exemplary recognition haptens, carboxyl-containing chelate derivatives such as DTPA, DOTA, HBED and HSG are readily coupled to amino-containing polymers by controlled activation of a limited number of their carboxylate groups using carbodiimide-like reagents. Fluorescein is readily coupled to amine-containing polymers using an isothiocyanate-derivatized analog. Similarly, amino-containing recognition haptens are readily coupled to suitable carboxyl-activated polymers having a plurality of free carboxyl groups. Haptens having aldehyde or ketone groups, or hydrazinyl or amino groups, are readily attached to polymers with the complementary functionality, with a reduction step to reduce Schiff-base type intermediates, optionally included. Haptens having a free thiol are readily attached to polymers using alyklation of activated halegeno-intermediates to give thioethers, by Michael addition, or by disulfide bond formation. Reverse wise, the free thiol can be on the polymer, and the electrophile on the hapten. Free hydroxy groups on haptens can be attached as ethers or esters, starting with a suitably derivatized polymer, while active hydrogens can be used in Mannich-type condensation reactions.

Some of the above discussion related to examples of polymer conjugates and haptens, apply equally generally to drugs to be used in the invention. For instance, part of the above discussion mentioned certain drugs, such as SN-38, the active metabolite of the prodrug CPT-11 (irinotecan). SN-38 has an aromatic hydroxyl group that was used in the above descriptions to produce aryl esters susceptible to esterase-type enzymes. Similarly the camptothecin analogs topotecan and 10-hydroxycamptothecin, used in chemotherapy, both have an available aromatic hydroxyl residue that can be used in a similar manner as described for SN-38, producing esterase-susceptible polymer-prodrugs. Also in this class can be placed taxol and certain Vinca alkaloids. In each instance, a drug containing a free hydroxyl group is attached to the polymer using an ester linkage. A preferred advantage of using an ester linkage is that the drug-polymer bond is cleavable, and whether localized intra- or extra-cellularly, the free and active drug can be produced over time, to exert its effect at the target site. Doxorubicin also contains a hydroxyl group that can be coupled to carboxylate-containing polymeric conjugates using acid-catalyzed reactions similar to those described for the camptothecin family.

Doxorubicin and other drugs with amino ‘chemical handles’ active enough for chemical coupling to polymer conjugates can be effectively coupled to conjugates via these free amino groups in a number of ways. Polymers bearing free carboxylate groups can be activated in situ (EDAC; water-soluble carbodiimide) and the activated polymers mixed with doxorubicin to directly attach the drug to the side-chains of the polymer via amide bonds. Amino-containing drugs can also be coupled to amino-pendant polymers by mixing a commercially available, and cleavable cross-linking agent such as ethylene glycobis(succinimidylsuccinate) (EGS, Pierce Chemical Co., Rockford, Ill.) or bis-[2-(succinimidooxycarbonyloxy)ethyl]sulfone (BSOCOES, Molecular Biosciences, Huntsville, Ala.) to cross-link the two amines as two amides after reaction with the bis(succinimidyl) ester groups. A preferred conjugate is one that, for instance, like (doxorubicin-EGS)ₙ-poly-lysine remains susceptible to enzymatic cleavage of the diester groups in the EGS linking chain by enzymes such as esterases.

Numerous analogs of doxorubicin have been prepared, and many of these can also be coupled to polymers in similar ways. Exemplary of these analogs is 2-pyrrolinodoxorubicin (2-PDOX), which has been reported to be 100-1000 more toxic to tumor cells than doxorubicin itself. With 2-PDOX, the free amino group present in doxorubicin has been converted into an enamine. However, at the other end of the 2-PDOX molecule a ketone group and a hydroxy group are available for conjugation reactions. The hydroxy group can be coupled with an activated poly-glutamic acid or poly-aspartic acid polymer, or a co-polymer containing a plurality of these sub-units, using standard esterification chemistry to produce ester-linked drug-polymer conjugates. In an alternative approach, poly acidic conjugates can be partially converted to hydrazides using standard coupling chemistries, and the hydrazides can later be condensed with the ketone groups in doxorubicin or 2-PDOX. The formed Schiff bases can be used without reduction, or can be reduced by short reaction with sodium borohydride or sodium cyanoborohydride.

Other well-known drugs, such as methotrexate, also have an available amino group for coupling to activated carboxylate-containing polymers, in a similar manner to that described for doxorubicin, above. Methotrexate also has two glutamyl carboxyl groups (alpha and gamma) that can be activated for coupling to amino-group containing polymers. The free carboxylate groups of methotrexate can be activated in situ (EDAC) and the activated drug mixed with an amino-containing polymer to directly attach the drug to the side-chains of the polymer via amide bonds. As with most conjugations of low MW materials to polymers, excess unreacted or cross-reacted drug are separated readily from the polymer-drug conjugate using size-exclusion or ion-exchange chromatography, or using dialysis or diafiltration.

Maytansinoids and calicheamicins (such as esperamycin) can also be used within the scope of the invention. The latter contain mixed di- and tri-sulfide bonds that can be cleaved to generate species with a single thiol useful for chemical manipulation. A copolymer such as polyGlu.Lys-OH s treated with a succinimidyl/maleimido cross-linking agent such that the linker adds to the free lysine amino groups of the polymer. This generates a multiplicity of malemimido residues on the polymer that can be reacted to varying substitution ratios with the thiol-containing drugs. The cross-linker used can be chosen to be susceptible to cleavage by peptidases. More preferably, drugs such as calicheamicins are linked to the polymer via a disulfide bond so that they are readily activated upon reduction. Thus co-polymers containing thiol residues such as cysteine are contemplated. In this preferred embodiment it can be emphasized that the reductive process needed to trigger the calicheamicin reaction cascade that leads to anti-growth activity, is much more likely to occur in the highly reductive intracellular compartment, as opposed to in the extra-cellular milieu. Hence multi-specific targeting agents that are inductively internalized upon cross-linking by the recognition hapten-polymer-drug conjugate are especially preferred. A key general issue is that whereas the drug-to-polymer linkage can be varied in terms of chemical bond stability, the link between the polymer backbone and the recognition hapten should be strong, and impervious to serum decomposition.

Even drugs classed as alkylating agents are within the scope of the invention, since chloro, bromo, tosyl, or mesyl mustard derivatives containing, for instance, a free carboxyl group can be coupled to polymers using active esters, azide or acid chloride intermediates, while leaving the alkylating moieties essentially intact. Alternatively, precursors of mustard agents can be coupled to polymers and the precursors, converted to the active agents by halogenation or analogous reactions.

The peptides to be used as polymers or recognition haptens are synthesized conveniently on an automated peptide synthesizer using a solid-phase support and standard techniques of repetitive orthogonal deprotection and coupling. Free amino groups in the peptide, that are to be used later for chelate conjugation, are advantageously blocked with small organic moieties, for example by acetylation. For instance, Ac-Gly-D-Tyr-D-Trp-Gly-D-Lys(Ac)-Gly-D-Tyr-D-Trp-OH, cleaved from its assembly resin is then activated through its single carboxyl moiety using active ester/anhydride methodology and coupled in multiple units to KLH. For immunogenic use, the di-cysteinyl-containing peptide Ac-Cys(Y)-D-Tyr-D-Trp-Gly-D-Cys(Y)-Gly-D-Tyr-D-Trp-OH can be removed from the resin with the thiol groups protected by methylation to generate Ac-Cys(Me)-D-Tyr-D-Trp-Gly-D-Cys(Me)-Gly-D-Tyr-D-Trp-OH. This can then be activated for KLH coupling using the same standard methods. When the peptides are prepared for later use within the msAb system, they are advantageously cleaved from the resins to generate the corresponding C-terminal amides, which will inhibit in vivo carboxypeptidase activity.

The components separately described in the above discussions are then applied to the treatment of subjects using the following general approaches. First, the optimum dose of multi-specific antibody is determined empirically and can be expressed in terms of mg of protein per kg or per square meter of subject. Second, the timing and dose of the hapten-polymer-agent conjugate that results in the optimum dose of drug being administered to the tumor target is determined, again, empirically, after pretargeting with the optimum dose of the multi-specific antibody. Determinations of optima such as these are readily made using standard techniques in pharmacology and radiopharmacology. Once the basic doses and timings have been determined, the method is ready to be applied more generally.

### EXAMPLES

All references cited herein are hereby incorporated herein by reference in their entireties.

### Example 1

Preparation of poly-glutamic acid (SN-38-ester)₁₀

A 10 g (2-6.66×10˜mole) amount of poly-glutamic acid (15-50 kDalton; Sigma Chemical Company) is mixed with 200 mL of dry, distilled dimethylformamide (DMF) and 3.92 g (1×10⁻² mole) SN-38. By means of a hydrogen chloride generator system (slow mixing of hydrochloric and concentrated sulfuric acid and passage of the resulting gas through concentrated sulfuric acid) dry hydrogen chloride gas is added to the DMF until a weight increase of 5 g has occurred. The mixture is heated under reflux for three hours using a Soxhlet extractor filled with dry magnesium sulfate to remove water from the DMF prior to its return to the reaction vial. After cooling, the product, poly-glutamic acid (SN-38-(-ester)₁₀, in DMF is treated with a ten-fold excess of diethyl ether to DMF. The collected precipitate is washed with ether, taken up in water and the aqueous solution extracted with chloroform to remove residual free SN-38. Poly-glutamic acid (SN-38-(-ester)₁₀ is collected as a lyophilized solid and the SN-38-to-polymer ratio determined spectrophotometrically.

### Example 2

Preparation of AcLys(HSG)Glu₆[10-hydroxycamptothecin]₆Lys[HSG]NH₂

The title peptide is prepared as a discrete entity using standard solid-phase synthetic methods with the first resin-appended lysine residue substituted with an allyloxycarbonyl epsilon protecting group and the last [N-terminus] lysyl residue protected similarly at its epsilon amino position. The N-terminus is acetylated at the conclusion of the chain synthesis. The glutamic acid gamma carboxyl groups are protected as the tert.-butyl esters during peptide assembly. Alpha amino groups are protected using Fmoc and the peptide is prepared on solid phase with successive rounds of Fmoc deprotection and coupling. Then the two-epsilon Aloc groups are selectively removed. The partially protected peptide is reacted with an excess of trityl-HSG through its free carboxyl group, using appropriate carboxyl group activation agents. Finally, the tert-butyl protecting groups are removed from the lysyl along with trityl- from the HSG residues, respectively, and the peptide cleaved from the resin, with trifluoroacetic acid. The 10-hydroxycamptothecin and the HSG-containing peptide are reacted together in toluene using acid catalysis and Dean-Stark conditions to produce AcLys(HSG)Glu₆[10-hydroxycamptothecin]₆Lys[HSG]NH₂ with the 10-hydroxycamptothecin moieties linked to the peptide glutamate gamma carboxyl groups as esters.

### Example 3

Coupling of Doxorubicin to a Random Co-Polymer of Glutamic Acid

A 70-150 kD random co-polymer comprised of poly(Glu,Glu-OMe), 4:1, is treated with an excess of hydrazine hydrate and allowed to stand for 24 h at room temperature. The excess hydrazine is removed by repeated dialysis, and the poly(Glu,Glu-NHNH₂) is mixed with a 5× excess (to estimated hydrazide) of doxorubicin, at a pH of 5. The mixture is allowed to stir overnight, and the excess doxorubicin is removed by repeated dialyses. The extent of doxorubicin substitution is determined spectrophotometrically. Optionally, the Schiff bases linking the doxorubicin to the polymer are reduced by overnight reaction with excess sodium cyanoborohydride.

### Example 4

Coupling of Calicheamicin to Metallothionein-DOTA

A solution of DOTA is activated in a limited manner, to activate only one of the four free carboxyl units available, by reaction with a ten-fold deficit of N-hydroxy-sulfo-succinimide, and a 100×deficit of the carbodiimide coupling agent, EDAC. A sample of metalbothionein is then dialyzed into phosphate buffer, pH 8, and treated with a solution of the activated DOTA chelating agent, in 10×molar excess to metallothionein. The coupling reaction is allowed to proceed overnight at four degrees Celsius, and the metallothionein-DOTA is purified from unreacted side-products by repeated dialyses against metal-free 0.2 M ammonium acetate buffer, pH 6, containing 1 mM EDTA. The DOTA-metalbothionein conjugate is then mixed with a ten-fold excess (to estimated free thiol groups) of calicheamicins, allowed to react overnight at four degrees Celsius, and repurified from unconjugated drug by repeated dialyses against metal-free 0.2 M ammonium acetate buffer, pH 6.

### Example 5

Coupling of an Alkylating Drug to a DTPA-Polymer Conjugate

A random copolymer of 2-(dimethylamino)ethyl methacrylate (DMAEMA) and aminoethyl methacrylate is produced by radical polymerization with animonium peroxydisulfate as radical initiator (60° C., 16 h). The p[DMAEMA]-co-AEMA] product, containing free primary amino groups, is purified by extensive dialysis against water. It is treated with a limited amount of DTPA dianhydride, in phosphate buffer, pH 8, and again dialyzed against water to remove unattached DTPA. The DTPA substitution ratio is estimated by titrating aliquots with increasing amounts of In-111 spiked cold indium chloride solution. The chemotherapy drug, chlorambucil, is mixed with an equimolar amount of dicyclohexylcarbodiimide and N-hydroxysuccinimide in dry dioxane. After stirring for 2 h, the formed dicyclohexylurea is filtered off. The dioxane solution of the ester-activated chlorambucil is added, in molar excess to available free amino groups, to the DTPA-p[DMAEMA]-co-AEMA] polymer in phosphate buffer, pH 8, to a final dioxane concentration up to 50%. After a 30-minute reaction the pH is adjusted to 3 with hydrochloric acid, and the chlorambucil-p[DMAEMA]-co-AEMA]-DTPA polymer is purified by filtration from insoluble drug, and by repeated dialyses against sodium acetate buffer, pH 4.

### Example 6

Development of an Antibody Against a Polyglutamate Polymer

Polyglutamic acid (average MW 15,000) is treated with a 20-fold molar deficit of the water-soluble carbodiimide EDAC, at pH 4, in the presence of a 20-fold molar deficit (to poly E) of N-hydroxysulfosuccinimide. The reaction is allowed to proceed for 3 h at room temperature, and the crude mixture is then added to a solution of KLH in phosphate buffer, pH 8. The product is purified by repeated dialyses against PBS. It is used for repeated injection into immunocompetent mice, initially with complete Freund's adjuvant and later with in complete Freund's adjuvant. To measure the immune response in terms of antibody titer, the same polyglutamate may be coupled to a non-specific IgG molecule (as a carrier) for better plate absorption, or absorbed directly onto an ELISA plate itself. A number of mice having good antibody titers are selected and splenocytes from these animals are fused with the mouse myeloma cell line SP2/0 according to the standard technique. Up to 3000 clones are screened by ELISA for reactivity with polyglutamate. Those clones identified as secreting an IgG that binds to polyglutamate are sub-cloned, and positive hybrids selected and adapted to grow in serum-free media. The IgG is produced in quantity using standard methods of cell culture, and can be coupled as an IgG or fragmented to F(ab′)₂ and Fab′ and then coupled to a suitable targeting vector, in a similar manner to that used for the anti-DTPA antibody described in detail below.

### Example 7

Development of an Antibody Against Doxorubicin

Doxorubicin (54.3 mg; 1×10⁻⁴ mole) is mixed with a two-fold molar excess of the cross-linker bis[sulfosuccinimidyl]suberate (BS³; Pierce Chemical Co., Rockford, Ill.; 114.4 mg; 2×10⁻⁴ mole) The activation is allowed to proceed for 30 minutes at room temperature, and the crude mixture is then added to a solution of 100 mg of bovine serum albumin in phosphate buffer, pH 8. The product, [doxorubicin]₁₀-BSA is purified by repeated dialyses against PBS, and used for repeated injection into immunocompetent mice, initially with complete Freund's adjuvant and later with incomplete Freund's adjuvant. To measure the immune response toward doxorubicin, in terms of antibody titer, the drug may be coupled to ovalbumin as a non-specific carrier protein, and the latter conjugate used for testing sera. A number of mice having good antibody titers are selected and splenocytes from these animals are fused with the mouse myeloma cell line SP2/0 according to the standard technique. Up to 3000 clones are screened by ELISA for reactivity with doxorubicin-ovalbumin. Those clones identified as secreting an IgG that binds to doxorubicin are sub-cloned, and positive hybrids selected and adapted to grow in serum-free media. The anti-doxorubicin IgG is produced in quantity using standard methods of cell culture, and can be coupled as an IgG or fragmented to F(ab′)₂ and Fab′ and then coupled to a suitable targeting vector, in a similar manner to that used for the anti-DTPA antibody described in detail below.

### Example 8

Preparation of an Anti-CEA×Anti-DTPA Bi-Specific Antibody

a) Introducing a maleimide group into IgG: A 1 mL solution of hMN-14 IgG (8.45 mg /mL) is pH adjusted with ˜3 uL of 1 N HCl to pH 7.2. To this is added 10.7 uL of a 10 mM aqueous solution of sulfo-SMCC (1.9 fold molar excess), and the reaction mixture is left at the room temperature for 45 min. Purification is done by size-exclusion chromatography on Sephadex G50/80 in 0.1 M sodium phosphate, pH 6.5. The protein concentration (A₂₈₀) and the maleimide content are determined (0.93 maleimides/IgG being found under this set of conditions). The latter determination involves reaction with a known excess of 2-mercaptoethanol (2-ME), followed by back titration of unconsumed 2-ME by Ellman's assay.

b) Reduction of 734 F(ab′)₂ to 734 Fab′: The F(ab′)₂ fragment of the 734 MAb (1.25 mL; 10 mg) is mixed with 0.1 mL of 100 mM cysteine in 20 mM HEPES buffer, pH 7.3. The buffer also contains 150 mM sodium chloride and 10 mM EDTA, and is flushed with argon to prevent premature re-oxidation of reduced disulfide bonds. The reaction is incubated at 37° C. for 50 minutes, and purified by size-exclusion chromatography using Sephadex G50/80 in 0.1 M phosphate/5 mM EDTA, pH 6.5, as running buffer.

c) Conjugation of the hMN-14-IgG and the 734-Fab′ fragment: HMN-14-maleimide and 734 Fab′, from the above reactions, are mixed in a 1:1 molar ratio. The reaction mixture is flushed with argon, and incubated at the room temperature for ˜1 h (50 mm—1.5 h can be successfully used in different runs). At the end of the reaction a 40-fold molar excess of N-ethylmaleimide (using 2.64 mM aq solution), is added and the mixture is left overnight at 4° C., to block excess thiol groups on the Fab′ fragment. The reaction mixture is purified using size-exclusion chromatography on Sephadex G50/80 and 0.1 M sodium phosphate pH 7.3, buffer.

d) Purification of the IgG×Fab′ msAb: A column (0.9 cm outside diameter) is filled with 3 mL of Affigel-DTPA gel, and is used to separate unconjugated IgG from DTPA-containing entities. The crude reaction product from c) above, is passed slowly through the Affigel-DTPA column, previously equilibrated in 0.1 M sodium phosphate buffer, pH 7.3. Unconjugated hMN-14 passes straight through the column. Bound fractions are eluted from the affinity column using 1 M EDTA, pH 4.0, and the combined eluates from the column this process are immediately pooled, and dialyzed against 0.2 M sodium phosphate pH 6.8, with 3 buffer changes. The sample is then concentrated and purified by preparative size-exclusion HPLC on a TSK G3000SW column using 0.2 M sodium phosphate pH 6.8 as eluent. Fractions containing monomeric species corresponding to the MW of IgG×Fab′ are pooled, and concentrated. Using this methodology at this scale, 7.7 mg of the conjugate is obtained (21.4% overall yield). The product shows a single peak on HPLC, while MALDI mass spectral analysis showed an average MW of 196803 (0.2% error rate).

### Example 9

Preparation of a Hormone-Antibody Targeting Agent

The somatostatin analog DTPA-octreotide is treated with an equivalent of each of the carbodiimide EDAC and N-hydroxy-sulfo-succinimide at pH 4. The mixture is allowed to stir for 2 h, and added in 20×molar excess to the antibody 679 IgG (anti-HSG) in phosphate buffer pH 8.5. After being allowed to react overnight at 4° C., the substituted 679 MAb is purified by repeated dialysis against phosphate buffered saline. The substitution ratio of the DTPA-octreotide onto the 679 MAb is determined by MALDI-TOF mass spectroscopy. The agent is useful against tumors expressing large numbers of somatostatin receptors.

### Example 10

Treatment of a Subject Expressing a CEA-Positive Tumor

A subject who has colon cancer that expresses the CEA antigen is given a 100 mg/m² dose of the bi-specific antibody hMN-14×734 F(ab′)₂×Fab′. After 24 hours, the subject is then given an equimolar dose of the indium complex of the AcLys(DTPA)Glu₆[SN-38]₆Lys[DTPA]NH₂ DTPA-polymer-drug, conjugate, previously prepared using the method of example 2, above. The DTPA-polymer-drug is localized selectively at the tumor due to the pretargeting with the msAb, causing a high concentration of the active agent SN-38 to also be localized. Over time, free SN-38 is released from the localized conjugate, exerting a therapeutic effect on the tumors.

### Example 11

Preparation of Metallothionein-(HSG)₂

A solution of histamine-succinyl-glycine (HSG) is activated by a four-hour reaction with a molar equivalent of N-hydroxy-sulfo-succinimide and a molar equivalent of EDC at pH 4 in aqueous solution. A sample of metallothionein previously dialyzed into phosphate buffer, pH 8, is treated with a solution of the activated HSG, in 5×molar excess to metallothionein. The coupling reaction is allowed to proceed overnight at four degrees Celsius, and the metallothionein-(HSG)₂ is purified from unreacted side-products by repeated dialyses against metal-free 0.2 M sodium acetate buffer, pH 4.5. The metallothionein-(HSG)₂ conjugate is then compounded for later reductive radiolabeling with rhenium-188 by making the solution 800 mg/mL in stannous ion containing a 100×excess (to tin) of sodium glucoheptonate, aliquoting into vials in 1-10 mg portions, freezing over dry ice, lyophilizing, and septum-sealing the vials under vacuum or semi-vacuum with argon.

### Example 12

Radiolabeling of Metallothionein-(HSG)₂ with Rhenium-188

A 10-mg sample of metallothionein-(HSG)₂ prepared and compounded for radiolabeling with rhenium-188, as described in example 15, is reconstituted with a 2-mL fraction (100 mCi) of rhenium-188 radionuclide in physiological saline, freshly eluted from a tungsten-188/rhenium-188 tandem generator system. The solution containing the metallothionein-(HSG)₂ and the rhenium-188 mixture is heated for 30 minutes at 95 degrees Celsius to effect reduction of the rhenium-188 from the +7 perrhenate form to a form that can be bound by the multiple free thiol groups of the metallothionein-(HSG)₂ polypeptide. Incorporation of rhenium-188 into metallothionein-(HSG)₂ is >90%.

### Example 13

Targeting Using a BisAb and Rhenium-188-Metallothionein(HSG)₂

A patient presenting with a tumor that expresses the antigen termed colon specific antigen p (CSA-p) is treated with 200 mg of the bisAb Mu9×679 IgG×Fab′ [anti CSA-p×anti-HSG]. One week later, when the amount of bisAb remaining in circulation has dropped below 5% ID/g, and while amounts remaining in tumor deposits remain high, the patient is treated with 100 mCi of rhenium-188-metallothionein-(HSG)₂, prepared as described in example 16, above. Recognition of the HSG moieties on the rhenium-188-metallothionein-(HSG)₂ by the Mu9×679 bisAb pretargeted via tumor antigen enables the specific delivery of the therapeutic rhenium-188 radionuclide to tumor sites.

### Example 14

Preparation of Indium-DTPA-poly(Glu.Tyr) [4:1]

A solution of poly(Glu.Tyr) [4:1] in 0.2 M sodium borate buffer pH 8.5 is treated with an excess of DTPA-dianhydride. The rapid reaction either results in substitution of DTPA onto the alpha-amino group of the co-polymer, or aqueous hydrolysis of the added anhydride groups. The DTPA-appended product is purified from low molecular weight materials by repeated dialyses against water and ammonium acetate buffer, pH 4.5. Prior to the final dialysis a three-fold molar excess of indium acetate is added to the DTPA-poly(Glu.Tyr) [4:1] intermediate, which is allowed to stir for an hour prior to said final dialysis. The product is obtained pure after evaporation and lyophilization of the remaining water and buffer components.

### Example 15

Preparation of Iodine-131-[indium-DTPA]-poly(Glu.Tyr) [4:1]

The indium-DTPA-poly(Glu.Tyr) [4:1] intermediate from example 18 is added to an Iodogen™ vial along with 0.3 M phosphate buffer, pH 6.0, and 200 mCi of iodine-131 radionuclide. The reaction is shaken for 15 minutes at room temperature, and the radioiodinated indium-DTPA-poly(Glu. Tyr) [4:1] transferred out of the Iodogen™ vial into a clean vial, where ascorbic acid is added to stop any further oxidative reaction. Unreacted iodine-131 is removed, if required, though an anion-exchange cartridge. The product, iodine-131-[indium-DTPA]-poly(Glu.Tyr) [4:1] is then ready for use.

### Example 16

Targeting Using a BisAb and Iodine-131-[indium-DTPA]-poly(Glu.Tyr) [4:1]

A patient presenting with a tumor that expresses the antigen termed epithelial glycoprotein (EGP) is treated with 200 mg of the bisAb RS7×734 F(ab′)₂×Fab′ [anti EGP×anti-indium-DTPA]. Four days later, when the amount of bisAb remaining in circulation drops below 5% ID/g, and while amounts remaining in tumor deposits stay high, the patient is treated with 150 mCi of iodine-131-[indium-DTPA]-poly(Glu.Tyr) [4:1], prepared as described in example 19, above. Recognition of the indium-DTPA moieties on the iodine-131-[indium-DTPA]-poly(Glu.Tyr) [4:1] by the RS7×734 bisAb pretargeted via tumor antigen enables the specific delivery of the therapeutic radionuclide iodine-131 to sites of tumor.

Additional references of interest include the following:

Bamias, A., and Epenetos, A. A. Two-step strategies for the diagnosis and treatment of cancer with bioconjugates. Antibody, Immunoconjugates, Radiopharm. 1992; 5: 385-395.

Barbet, J., Peltier, P., Bardet, S., Vuillez, J P., Bachelot, I., Denet, S., Olivier, P., Lecia, F., Corcuff, B., Huglo, D., Proye, C., Rouvier, E., Meyer, P., Chatal, J. F. Radioimmunodetection of medullary thyroid carcinoma using indium-111 bivalent hapten and anti-CEA×anti-DTPA-indium bispecifc antibody. J. Nucl. Med. 1998; 39:1172-1178.

Bos, E S., Kuijpers, W H A., Meesters-Winters, M., Pham, D T., deHaan, A S., van Doormalen, Am., Kasperson, F. M., vanBoeckel, C A A and Gouegeon-Bertrand, F. In vitro evaluation of DNA-DNA hybridization as a two-step approach in radioimmunotherapy of cancer. Cancer Res. 1994; 54:3479-3486.

Gautherot, E., Bouhou, J., LeDoussal, J -M., Manetti, C., Martin, M., Rouvier, E., Barbet, J. Therapy for colon carcinoma xenografts with bi-specific antibody-targeted, iodine-131-labeled bivalent hapten. Cancer suppl. 1997; 80: 2618-2623.

Gautherot, E., Bouhou, J., Loucif, E., Manetti, C., Martin, M., LeDoussal, J. M., Rouvier, E., Barbet, J. Radioimmunotherapy of LS174T colon carcinoma in nude mice using an iodine-131-labeled bivalent hapten combined with an anti-CEA×anti-indium-DTPA bi-specific antibody. J. Nucl. Med. Suppl. 1997; 38: 7p.

Goodwin, D. A., Meares, C F., McCall, M J., McTigue, M., Chaovapong, W. Pre-targeted immunoscintigraphy of murine tumors with indium-111-labeled bifunctional haptens. J. Nucl. Med. 1988; 29:226-234.

Greenwood, F. C. and Hunter, W. M. The preparation of 1-131 labeled human growth hormone of high specific radioactivity. Biochem. 1963; 89:114-123.

Hawkins, G. A., McCabe, R. P., Kim, C. -H., Subramanian, R., Bredehorst, R., McCullers, G. A., Vogel, C. -W., Hanna, M. G. Jr., and Pomata, N. Delivery of radionuclides to pretargeted monoclonal antibodies using dihydrofolate reductase and methotrexate in an affinity system. Cancer Res. 1993; 53: 2368-2373.

Kranenborg, M. h., Boerman, O. C., Oosterwijk-Wakka, j., weijert, M., Corstens, F., Oosterwijk, E. Development and characterization of anti-renal cell carcinoma×antichelate bi-specific monoclonal antibodies for two-phase targeting of renal cell carcinoma. Cancer Res.(suppl) 1995; 55: 5864s-5867s

Penefsky, H. S. A centrifuged column procedure for the measurement of ligand binding by beef heart F1. Part G. Methods Enzymol. 1979; 56:527-530.

Schuhmacher, J., Klivenyi, G., Matys, R., Stadler, M., Regiert, T., Hauser, H., Doll, J., Maier-Borst, W., Zoller, M. Multistep tumor targeting in nude mice using bi-specific antibodies and a gallium chelate suitable for immunocintigraphy with positron emission tomography. Cancer Res. 1995; 55, 115-123.

Sharkey, R M., Karacay, Griffiths, G L., Behr, T M., Blumenthal, R D., Mattes, M J., Hansen, H J., Goldenberg. Development of a streptavidin-anti-carcinoembryonic ntigen antibody, radiolabeled biotin pretargeting method for radioimmunotherapy of colorectal cancer. Studies in a human colon cancer xenograft model. Bioconjugate Chem 1997; 8:595-604.

Stickney, D R., Anderson, L D., Slater, J B., Ahlem, C N., Kirk, G A., Schweighardt, S A and Frincke, J M. Bifunctional antibody: a binary radiopharmaceutical delivery system for imaging colorectal carcinoma. Cancer Res. 1991;51: 6650-6655.

All of the publications and patent applications and patents cited in this specification are herein incorporated in their entirety by reference.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention, which is defined by the following claims.

What is claimed is:

1. A method for diagnosing or treating a disease or disorder comprising:
(a) administering to a tissue a multi-specific antibody or antibody fragment, comprising a targeting arm that binds to an antigen on said target site, and a capture arm that binds to a polymer conjugate; and
(b) administering to said tissue a polymer conjugate that binds to said capture arm, said polymer conjugate comprising a polymer conjugated to a diagnostic or therapeutic agent.

2. The method of claim 1, wherein said disease or disorder is selected from the group consisting of a cancer, cardiovascular lesion, an inflammatory disease, neurodegenerative disease, metabolic disease, and an infectious disease.

3. The method of claim 2, wherein said cancer is selected from the group consisting of a solid tumor, a B-cell malignancy and a T-cell malignancy.

4. The method of claim 3, wherein said disease or disorder is a B-cell malignancy selected from the group consisting of indolent forms of B-cell lymphomas, aggressive forms of B-cell lymphomas, chronic lymphatic leukemias, acute lymphatic leukemias, and multiple myeloma.

5. The method of claim 3, wherein said solid tumor is selected from the group consisting of a melanoma, carcinoma, glioma and sarcoma.

6. The method of claim 5, wherein said carcinoma is selected from the group consisting of renal carcinoma, lung carcinoma, intestinal carcinoma, and stomach carcinoma.

7. The method of claim 2, wherein said cancer is selected from the group consisting of esophageal, gastric, colonic, rectal, pancreatic, lung, breast, ovarian, urinary bladder, endometrial, cervical, testicular, renal, adrenal and liver cancer.

8. The method of claim 2, wherein said cardiovascular lesion is selected from the group consisting of an infarct, clot, embolus, atherosclerotic plaque, and ischemia.

9. The method of claim 2, wherein said neurodegenerative disease is Alzheimer's disease.

10. The method of claim 2, wherein said metabolic disease is amyloidosis and said antibody binds amyloid.

11. The method of claim 1, wherein said disease or disorder is displaced or ectopic normal tissue.

12. The method of claim 11, wherein said tissue is selected from the group consisting of endometrium, thymus, spleen and parathyroid.

13. The method of claim 1, wherein said method can be used for normal tissue ablation.

14. The method of claim 11, wherein said tissue is selected from the group consisting of bone marrow and spleen.

15. The method of claim 1, wherein said disease or disorder is an autoimmune disease.

16. The method of claim 15, wherein said autoimmune disease is selected from the group consisting of myasthenia gravis, lupus nephritis, lupus erythematosus, and rheumatoid arthritis, Class III autoimmune diseases such as immune-mediated thrombocytopenias, such as acute idiopathic thrombocytopenic purpura and chronic idiopathic thrombocytopenic purpura, dermatomyositis, Sjögren's syndrome, multiple sclerosis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, sarcoidosis, ulcerative colitis, erythema multiforme, IgA nephropathy, polyarteritis nodosa, ankylosing spondylitis, Goodpasture's syndrome, thromboangitis ubiterans, Sjogren's syndrome, primary biliary cirrhosis, Hashimoto's thyroiditis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis and fibrosing alveolitis.

17. The method of claim 2, wherein said infectious disease is selected from the group consisting of a bacterial, fungal, parasitic and viral lesion.

18. The method of claim 17, wherein said infectious disease is caused by a fungus selected from the group consisting of Microsporum, Trichophyton, Epidermophyton, Ssporothrix schenckii, Cyrptococcus neoformans, Coccidioides immitis, Histoplasma capsulatum, Blastomyces dermatitidis, and Candida albicans.

19. The method of claim 17, wherein said infectious disease is caused by a virus selected from the group consisting of human immunodeficiency virus (HIV), herpes virus, cytomegalovirus, rabies virus, influenza virus, hepatitis B virus, Sendai virus, feline leukemia virus, Reo virus, polio virus, human serum parvo-like virus, simian virus 40, respiratory syncytial virus, mouse mammary tumor virus, Varicella-Zoster virus, Dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus and blue tongue virus.

20. The method of claim 17, wherein said infectious disease is caused by a bacterium selected from the group consisting of Anthrax bacillus, Streptococcus agalactiae, Legionella pneumophilia, Streptococcus pyogenes, Escherichia coli, Neisseria gonorrhoeae, Neisseria meningitidis, Pneumococcus, Hemophilis influenzae B, Treponema pallidum, Lyme disease spirochetes, Pseudomonas aeruginosa, Mycobacterium leprae, Brucella abortus, Mycobacterium tuberculosis and Tetanus toxin.

21. The method of claim 17, wherein said infectious disease is caused by a parasite selected from the group consisting of a helminth or a malarial parasite.

22. The method of claim 2, wherein said infectious disease is caused by a protozoa selected from the group consisting of Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japanicum, Babesia bovis, Elmeria tenella, Onchocerca volvulus, Leishmania tropica, Trichinella spiralis, Onchocerca volvulus, Theileria parva, Taenia hydatigena, Taenia ovis, Taenia saginata, Echinococcus granulosus and Mesocestoides corti.

23. The method of claim 2, wherein said infectious disease is caused by a mycoplasma selected from the group consisting of Mycoplasma arthritidis, Mycoplasma hyorhinis, Mycoplasma orale, Mycoplasma arginini, Acholeplasma laidlawii, Mycoplasma salivarum, and Mycoplasma pneumoniae.

24. The method of claim 1, wherein said antigen is selected from the group consisting of carcinoembryonic antigen (CEA), HER-2/neu, epidermal growth factor receptor (EGFR), VEGF, placental growth factor (PLGF), tenascin, EGP-1, EGP-2, CD19, CD20, CD22, CD21, CD23, CD30, CD33, CD45, CD80, and CD74. α-fetoprotein, A3, CA125, colon-specific antigen-p (CSAp), folate receptor, HLA-DR, human chorionic gonadrotropin, Ia, IL-2, insulin-like growth factor, KS-1, Le(y), MAGE, MUC1, MUC2, MUC3, MUC4, NCA66, necrosis antigens, PAM-4, prostatic acid phosphatase (PAP), Pr1, prostate specific antigen (PSA), PSMA, S100, T101, TAC, IL-6 and TAG-72.

25. The method of claim 2, wherein said cancer is selected from the group consisting of a CEA-expressing tumor or a CD20-expressing malignancy.

26. The method of claim 25, wherein said CD20-expressing malignancy is a B-cell lymphoma or leukemia.

27. The method of claim 1, wherein said polymer conjugate has a general formula comprising (polymer backbone)-(agent)ₘ, where m is an integer.

28. The method of claim 1, wherein said polymer conjugate further comprises a recognition hapten conjugated to said polymer.

29. The method of claim 1, wherein said polymer conjugate has a general formula comprising (recognition hapten)ₙ-(polymer backbone)-(agent)ₘ, where n and m are integers.

30. The method of claim 29, wherein said recognition hapten is selected from the group consisting of: diethylenetriaminepentaacetic acid (DTPA), a metal complex of DTPA, 1,4,7,10-tetrazacyclododecane-N,N′, N′″, N′″-tetraacetic acid (DOTA), a metal complex of DOTA, N,N′-di[2-hydroxy-5-(ethylene-∃-carboxy)benzyl]ethylenediamine N,N′-diacetic acid (HBED), a metal complex of HBED, fluorescein, 2,4-dinitrophenyl-derivatives, biotin and histaminyl-succinyl-glycine.

31. The method of claim 1, wherein said multi-specific antibody or antibody fragment is radiolabeled.

32. The method of claim 31, wherein said multi-specific antibody or antibody fragment is radiolabeled with a radionuclide selected from the group consisting of F-18, P-32, Sc-47, Cu-62, Cu-64, Cu-67, Ga-67, Ga-68, Y-86, Y-90, Zr-89, Tc-99m, Pd-109, Ag-111, In-111, I-123, I-125, I-131, Sm-153, Gd-155, Gd-157, Tb-161, Lu-177, Re-186, Re-188, Pt-197, Pb-212, Bi-212, Bi-213, Ra-223, Ac-225, As-72, As-77, At-211, Au-198, Au-199, Bi-212, Br-75, Br-76B, C-11, Co-55Co, Dy-166, Er-169, F-18, Fe-52, Fe-59, Ga-67, Ga-68, Gd -154-158, Ho-166, I-120, I-121, I-124, In-110, In-111, Iri194, Lu-177, Mn-51, Mn-52, Mo-99, N-13, O-15, P-32, P-33, Pb-211, Pb-212, Pd-109, Pm-149, Pr-142, Pr-143, Rb-82, Re-189, Rh-105, Sc-47, Se-75, Sr-83, Sr-89, Tb-161, Tc-94, Tc-99, Y-86, Y-90 and Zr-89.

33. The method of claim 1, further comprising administering a clearing composition to said tissue and allowing said clearing composition to clear unbound said multi-specific antibody or antibody fragment from said tissue.

34. The method of claim 1, wherein said multi-specific antibody or antibody fragment is a monoclonal antibody.

35. The method of claim 1, wherein said multi-specific antibody or antibody fragment is chimeric, humanized, or human.

36. The method of claim 1, wherein said polymer is selected from the group consisting of polymers of single amino acids, co-polymers of two amino acids, co-polymers of three amino acids, co-polymers of four amino acids, polyethylene glycol (PEG), derivatives of PEG, co-polymers of PEG, N-(2-hydroxypropyl)methacrylamide (HPMA), polystyrene-co-maleic acid/anhydride (SMA), polyvinylether maleic anhydride (DIVEMA), polyethyleneimine, ethoxylated ployethyleneimine, starburst dendrimers, polyvinylpyrrolidone (PVP), apometallothionein and calicheamicin.

37. The method of claim 1, wherein said therapeutic agent is selected from the group consisting of a therapeutic radioisotope, toxin, cytokine, immunomodulator, drug, prodrug and boron addend.

38. The method of claim 37, wherein said drug is selected from the group consisting of taxanes, nitrogen mustards, ethylenimine derivatives, alkyl sulfonates, nitrosoureas, triazenes; folic acid analogs, pyrimidine analogs, purine analogs, vinca alkaloids, antibiotics, enzymes, platinum coordination complexes, substituted urea, methyl hydrazine derivatives, adrenocortical suppressants, antagonists, steroids, progestins, estrogens, antiestrogens, androgens, azaribine, anastrozole, azacytidine, bleomycin, bryostatin-1, busulfan, carmustine, chlorambucil, cisplatin, irinotecan (CPT-11), carbopiatin, celebrex, cladribine, cyclophosphamide, cytarabine, dacarbazine, docetaxel, dactinomycin, daunorubicin, dexamethasone, diethylstilbestrol, doxorubicin, ethinyl estradiol, estramustine, etoposide, floxuridine, fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, methotrexate, mitoxantrone, mithramycin, mitomycin, mitotane, phenyl butyrate, prednisone, procarbazine, paclitaxel, pentostatin, semustine streptozocin, tamoxifen, taxanes, taxol, testosterone propionate, thalidomide, thioguanine, thiotepa, teniposide, topotecan, uracil mustard, vinblastine, vinorelbine or vincristine.

39. The method of claim 1, wherein said therapeutic or diagnostic agent is selected from the group consisting of radioisotopes, enhancing agents for use in magnetic resonance imaging, contrasting agents, and coloring agents.

40. The method of claim 37, wherein said cytokine is selected from the group consisting of IL-1, IL-2, IL-3, IL-6, IL-10, IL-12, IL-18, IL-21, interferon-α, interferon-β, interferon-γ, GM-CSF, G-CSF, erythropoietin and thrombopoietin.

41. A method for photodynamic diagnosis or treatment of a disease or disorder comprising:
(a) administering to a tissue a multi-specific antibody or antibody fragment, comprising a targeting arm that binds to an antigen on said target site, and a capture arm that binds to a polymer conjugate; and
(b) administering to said tissue a polymer conjugate that binds to said capture arm, said polymer conjugate comprising a polymer conjugated to a diagnostic or therapeutic agent.

42. The method of claim 41, wherein said therapeutic agent is a photosensitizer.

43. The method of claim 42, wherein said photosensitizer is selected from the group consisting of a dihematoporphyrin, benzoporphyrin monoacid ring A, tin etiopurpurin, sulfonated aluminum phthalocyanine, and lutetium texaphyrin.

44. An intravascular or endoscopic method for diagnosing or treating a disease or disorder comprising
(a) administering to a tissue a multi-specific antibody or antibody fragment, comprising a targeting arm that binds to an antigen on said target site, and a capture arm that binds to a polymer conjugate; and
(b) administering to said tissue a polymer conjugate that binds to said capture arm, said polymer conjugate comprising a polymer conjugated to a diagnostic or therapeutic agent.

45. The method of claim 44, wherein said disease or disorder is selected from the group consisting of a cancer, cardiovascular lesion, an inflammatory disease, neurodegenerative disease, metabolic disease, and an infectious disease.

46. The method of claim 45, wherein said cancer is selected from the group consisting of a solid tumor, a B-cell malignancy and a T-cell malignancy.

47. The method of claim 46, wherein said disease or disorder is a B-cell malignancy selected from the group consisting of indolent forms of B-cell lymphomas, aggressive forms of B-cell lymphomas, chronic lymphatic leukemias, acute lymphatic leukemias, and multiple myeloma.

48. The method of claim 46, wherein said solid tumor is selected from the group consisting of a melanoma, carcinoma, glioma and sarcoma.

49. The method of claim 48, wherein said carcinoma is selected from the group consisting of renal carcinoma, lung carcinoma, intestinal carcinoma, and stomach carcinoma.

50. The method of claim 45, wherein said cancer is selected from the group consisting of esophageal, gastric, colonic, rectal, pancreatic, lung, breast, ovarian, urinary bladder, endometrial, cervical, testicular, renal, adrenal and liver cancer.

51. The method of claim 45, wherein said cardiovascular lesion is selected from the group consisting of an infarct, clot, embolus, atherosclerotic plaque, and ischemia.

52. The method of claim 45, wherein said neurodegenerative disease is Alzheimer's disease.

53. The method of claim 45, wherein said metabolic disease is amyloidosis and said antibody binds amyloid.

54. The method of claim 44, wherein said disease or disorder is an autoimmune disease.

55. The method of claim 54, wherein said autoimmune disease is selected from the group consisting of myasthenia gravis, lupus nephritis, lupus erythematosus, and rheumatoid arthritis, Class III autoimmune diseases such as immune-mediated thrombocytopenias, such as acute idiopathic thrombocytopenic purpura and chronic idiopathic thrombocytopenic purpura, dermatomyositis, Sjögren's syndrome, multiple sclerosis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, sarcoidosis, ulcerative colitis, erythema multiforme, IgA nephropathy, polyarteritis nodosa, ankylosing spondylitis, Goodpasture's syndrome, thromboangitis ubiterans, Sjogren's syndrome, primary biliary cirrhosis, Hashimoto's thyroiditis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis and fibrosing alveolitis.

56. The method of claim 45, wherein said infectious disease is selected from the group consisting of a bacterial, fungal, parasitic and viral lesion.

57. The method of claim 56, wherein said infectious disease is caused by a fungus selected from the group consisting of Microsporum, Trichophyton, Epidermophyton, Ssporothrix schenckii, Cyrptococcus neoformans, Coccidioides immitis, Histoplasma capsulatum, Blastomyces dermatitidis, and Candida albicans.

58. The method of claim 56, wherein said infectious disease is caused by a virus selected from the group consisting of human immunodeficiency virus (HIV), herpes virus, cytomegalovirus, rabies virus, influenza virus, hepatitis B virus, Sendai virus, feline leukemia virus, Reo virus, polio virus, human serum parvo-like virus, simian virus 40, respiratory syncytial virus, mouse mammary tumor virus, Varicella-Zoster virus, Dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus and blue tongue virus.

59. The method of claim 56, wherein said infectious disease is caused by a bacterium selected from the group consisting of Anthrax bacillus, Streptococcus agalactiae, Legionella pneumophilia, Streptococcus pyogenes, Escherichia coli, Neisseria gonorrhoeae, Neisseria meningitidis, Pneumococcus, Hemophilis influenzae B, Treponema pallidum, Lyme disease spirochetes, Pseudomonas aeruginosa, Mycobacterium leprae, Brucella abortus, Mycobacterium tuberculosis and Tetanus toxin.

60. The method of claim 56, wherein said infectious disease is caused by a parasite selected from the group consisting of a helminth or a malarial parasite.

61. The method of claim 45, wherein said infectious disease is caused by a protozoa selected from the group consisting of Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japanicum, Babesia bovis, Elmeria tenella, Onchocerca volvulus, Leishmania tropica, Trichinella spiralis, Onchocerca volvulus, Theileria parva, Taenia hydatigena, Taenia ovis, Taenia saginata, Echinococcus granulosus and Mesocestoides corti.

62. The method of claim 45, wherein said infectious disease is caused by a mycoplasma selected from the group consisting of Mycoplasma arthritidis, Mycoplasma hyorhinis, Mycoplasma orale, Mycoplasma arginini, Acholeplasma laidlawii, Mycoplasma salivarum, and Mycoplasma pneumoniae.

63. The method of claim 45, wherein said antigen is selected from the group consisting of carcinoembryonic antigen (CEA), HER-2/neu, epidermal growth factor receptor (EGFR), VEGF, placental growth factor (PLGF), tenascin, EGP-1, EGP-2, CD19, CD20, CD22, CD21, CD23, CD30, CD33, CD45, CD80, and CD74. α-fetoprotein, A3, CA125, colon-specific antigen-p (CSAp), folate receptor, HLA-DR, human chorionic gonadrotropin, Ia, IL-2, insulin-like growth factor, KS-1, Le(y), MAGE, MUC1, MUC2, MUC3, MUC4, NCA66, necrosis antigens, PAM-4, prostatic acid phosphatase (PAP), Pr1, prostate specific antigen (PSA), PSMA, S100, T101, TAC, IL-6 and TAG-72.

64. The method of claim 45, wherein said cancer is selected from the group consisting of a CEA-expressing tumor or a CD20-expressing malignancy.

65. The method of claim 45, wherein said CD20-expressing malignancy is a B-cell lymphoma or leukemia.

66. The method of claim 44, wherein said polymer conjugate has a general formula comprising (polymer backbone)-(agent)ₘ, where m is an integer.

67. The method of claim 66, wherein said polymer conjugate further comprises a recognition hapten conjugated to said polymer.

68. The method of claim 44, wherein said polymer conjugate has a general formula comprising (recognition hapten)ₙ-(polymer backbone)-(agent)ₘ, where n and m are integers.

69. The method of claim 68, wherein said recognition hapten is selected from the group consisting of: diethylenetriaminepentaacetic acid (DTPA), a metal complex of DTPA, 1,4,7,10-tetrazacyclododecane-N,N′,N″,N′″-tetraacetic acid (DOTA), a metal complex of DOTA, N,N′-di[2-hydroxy-5-(ethylene-∃-carboxy)benzyl]ethylenediamine N,N′-diacetic acid (HBED), a metal complex of HBED, fluorescein, 2,4-dinitrophenyl-derivatives, biotin and histaminyl-succinyl-glycine.

70. The method of claim 44, further comprising administering a clearing composition to said tissue and allowing said clearing composition to clear unbound said multi-specific antibody or antibody fragment from said tissue.

71. The method of claim 44, wherein said multi-specific antibody or antibody fragment is a monoclonal antibody.

72. The method of claim 44, wherein said multi-specific antibody or antibody fragment is chimeric, humanized, or human.

73. The method of claim 44, wherein said polymer is selected from the group consisting of polymers of single amino acids, co-polymers of two amino acids, co-polymers of three amino acids, co-polymers of four amino acids, polyethylene glycol (PEG), derivatives of PEG, co-polymers of PEG, N-(2-hydroxypropyl)methacrylamide (HPMA), polystyrene-co-maleic acid/anhydride (SMA), polyvinylether maleic anhydride (DIVEMA), polyethyleneimine, ethoxylated ployethyleneimine, starburst dendrimers, polyvinylpyrrolidone (PVP), apometallothionein and calicheamicin.
